(19) United States
(12) Patent Application Publication
(10) Pub. No.: US 2006/0014987 A1
(43) Pub. Date: Jan. 19, 2006

(54) Synthesis of beta-elemene, intermediates thereto, analogues and uses thereof

(76) Inventor: Lan Huang, Bronx, NY (US)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/886,334

(22) Filed: Jul. 7, 2004

Related U.S. Application Data
(60) Provisional application No. 60/485,358, filed on Jul. 7, 2003.

(51) Int. Cl. C07C 35/24

(52) U.S. Cl. 568/820

(57) ABSTRACT

The present invention provides convergent processes for preparing beta-elemene, and analogues thereof. Also provided are analogues related to beta-elemene and intermediates useful for preparing the same. The present invention further provides novel compositions based on analogues of beta-elemene and methods for the treatment of cancer, such as brain tumor, lung cancer, colorectal cancer, gastric intestional cancer, and stomach cancer.

### RELATED APPLICATIONS

This application claims priority from U.S. provisional application Ser. No. 60/485,358, filed 7 Jul. 2003.

### GOVERNMENT INTEREST

None.

### FIELD OF THE INVENTION

The present invention is in the field of elemene. In particular, the present invention relates to processes for the preparation of (−)-beta-elemene, and derivatives thereof which are useful as highly specific, non-toxic anticancer therapeutics, and its MDR effects. The present invention also provides novel compositions of matter which serve as intermediates for preparing the (−)-beta-elemene. This invention also covers the usage of (−)-beta-elemene and (−)-beta-elemene derivatives and (−)-beta-elemene-like structures.

The invention, however, is applicable to cancers generally in mammals and the reference to human biochemistry is not intended to be limiting, but illustrative. The term patient or body or reference to humans is utilized for convenience, but includes all mammalian patients or bodies.

### BACKGROUND OF THE INVENTION

### Introduction

(−)-beta-elemene was approved recently by the Chinese FDA for glioblastoma. The major active component is (−)-beta-Elemene (C₁₅H₂₄, chemical name: 1-melthyl-vinyl-2, 4-diisoprenaline, M.W. 200.4), which can pass the blood-brain-barrier. The longest survival time of a glioblastoma patient is 62 months after treatment with this drug. Our collaborator Yuanda International Group (Dalian, P. R. China) holds the US patent (Pat. No. 6,464,839) and the China rights for this drug.

(−)-Beta-elemene is a naturally occurring compound that can be isolated from many sources including G. Cymbopogon winterianus Jowitt, Zhangzhou Aglaia odorata flower, Fuzhou Aglaia odorata flower, Chunging Aglaia odorata flower, Chunging Aglia odorata leaves, Zhangzhou Aglaia odorata leaves, Yibin geranium leaves, Kunmin geranium leaves, Litchi chenensis cinnamomifolium, dry Lauris nobilis, Citrus limona leaves, Vitis vinifera grape leaves, Clausena lansium leaves, Fortunella margarita leaves, Fortunella odorata, C. Wenyunjin Chen, and Magnolia sieboldi. It was first extracted in 1983? Elemene drug is a mixture of Elemene stereomers, with beta form as its major component (>85%).? Yuanda has conducted animal tests on 98% pure of (−)-beta-Elemene, which exhibits similar clinical effects as that of 85% pure (−)-beta-Elemene. Elemene is a non-cytotoxic agent for tumor therapy. Elemene emulsion (0.5% emulsion injection) has been approved for the treatment of different types of cancer in China since 1994. It has been used to treat over 10,000 cancer patients and its efficacy/safety profiles are well documented in the Chinese medical literature.

Yuarida International Group has created a new formulation of Elemene (2% emulsion injection), which contains the same Active Pharmaceutical Ingredient (API), but different non-active components to stabilize Elemene in a clear solution. In 61 patient trials conducted in China, our Elemene drug (2% emulsion) is better than the available drugs on the market for brain tumor patients, with tumor shrinkage effect (CR+PR) in 35-40% of the patient group. Drug TEMODAR has a CR+PR rate of 20%.

Molecular Mechanism

Beta-elemene is a highly active anticancer compound isolated from the roots of Curcuma Wenyujin. The total synthesis of beta-elemene and its derivatives is an important goal for several reasons as below, explained in its molecular mechanism.

Elemene inhibits cancer cell growth/division, through blocking cell cycle transition from G0/G1 phase to S phase (Xu, X. J. et al. Studies of β-Elemene's induction of human liver cancer cells, Chinese Journal of Clinical Oncology, Jul. 30-32, 1999).

According to the flow cytometry data (Elemene at 20 ug/ml, liver cancer cell SMMC), Elemene blocked G0/G1 to S phase transition.

Immunocytochemistry data indicated that Elemene induced tumor suppresser p53's expression, which potentially leads to inhibition of G0/G1 to S phase transition for DNA repair (Xu, X. J. et al. Studies of β-Elemene's induction of human liver cancer cells, Chinese Journal of Clinical Oncology, Jul. 30-32, 1999).

Elemene induces apoptosis at dose and time dependent manner, according to electron microscopy and DNA fragmentation data (Xu, X. J. et al. Studies of β-Elemene's induction of human liver cancer cells, Chinese Journal of Clinical Oncology, Jul. 30-32, 1999).

Apoptosis induced by Elemene might be due to Elemene's effect on protein expression levels: decrease of Bcl-2 and c-myc, and elevation of p53.

Bcl-2 protein: Bcl-2 inhibits apoptosis. Bcl-2 protein is not expressed in normal liver cells, and its high expression could lead to tumor cell's survival.

c-myc is a signaling protein, preceding signal transduction pathways. o-Myc potentially induces cell division.

P53, a hallmark tumor suppresser is especially linked to apoptosis. When DNA is damaged in cells, p53 protein level increases to inhibit G0/G1 to S transition for DNA repair. When DNA could not be repaired, p53 induces apoptosis.

Elemene also induced apoptosis and down-regulates expression of Bcl-2 protein in human leukemia K562 cells (Yuan. J et al. Elemene induces apoptosis and regulates expression of bcl-2 protein in human leukemia K562 cells, Zhongguo Yao Li Xue Bao (Chinese Pharmacology Journal), 20: 103-106, 1999).

Elemene Induces Cancer Cell's Differentiation

Elemene induces differentiation of lung tumor cells (Aip-937, A549, SPC-A1, small cell lung cancer H128) (Qian, J. et al. The studies of Elemene Emulsion on the Reversion of human lung cancer cells, Chinese Journal of Clinical Oncology, Jul. 7-10, 1999), melanoma cells B16 (Qiang, j. Et al. The induction of Differentiation of B16 cells y Elemene Emulsion, Chinese Journal of Clinical Oncology, Jul. 16-19, 1999). The ultrastructure showed the morphological changes, such as microvilli decrement and nucleus pyknosis.

Elemene does not produce Multi-drug Resistance (MDR) effect (Wang, B. C. et al. The Experimental Studies of Association between Elemene and Tumor Multidrug Resistance, Chinese Journal of Clinical Oncology, Jul. 10-13, 1999).

Human hepatic cancer BEL-7402 cell line was cultured and its drug-resistance strain BEL-7402/DOX was established. After 6 weeks of induction with Elemene at 48.9 ug/ml, drug resistant BEL-7402 cells still did not express MDR1 mRNA or P-glycoprotein (P-gp). Thus drug-resistant tumor cells are sensitive to Elemene.

Elemene passes the blood brain barrier (BBB) (Qian, J., New anti-tumor drug, Elemene's pharmacology and Clinical results, Chinese Journal of Clinical Oncology, Jul. 1-3, 1999). ³H labeled Elemene was injected intravenously into or taken orally by experimental animals. Radioactivity was detected in animals' brain.

Overall, Elemene is different from other cytotoxic cancer drugs, with high IC₅₀ for tumor cells (at 20-50 ug/ml in vitro). Its clinical tumor shrinkage effect is mainly due to its ability to induce apoptosis, inhibit cell cycle, and induce differentiation.

Accordingly, the present inventors undertook the total synthesis of beta-elemene, and as a result, have developed efficient processes for beta-elemene, as well as derivatives thereof. Each of the published method are inadequate for the purpose of obtaining (−)-beta-elemene. The present invention also provides novel intermediates useful in the synthesis of beta-elemene and derivatives thereof, compositions derived from such beta-elemene and derivatives, purified compounds of beta-elemene and derivatives, in addition to methods of use of the beta-elemene and beta-elemene derivatives in the treatment of cancer. Remarkably, beta-elemene and its derivatives of the invention have exceptionally high specificity as anti-tumor agents in vivo, and are more effective for cancer treatment, and less toxic to normal cells than the principal chemotherapeutics currently in use, including taxol, vinblastin, adriamycin and camptothecin.

Brain Tumor Field's Need of New Drugs

Brain Tumor Introduction

The development of new effective brain tumor therapies is lag compared to the treatment of other malignancies, with prognoses and mortality rates similar to those from 30 years ago. Malignant gliomas, the most common subtype of primary brain tumors, are aggressive, highly invasive, and neurologically destructive tumors. Its most aggressive manifestation is glioblastoma, with median survival ranges from 9 to 12 months, despite maximum treatment efforts.

15,000 brain tumor cases are reported each year in the United States. Since more than 50% of these tumors are malignant gliomas, upwards of 7,500 new cases of glioblastoma and anaplastic astrocytoma can be expected to occur yearly. Brain tumors are the second leading cause of cancer death in children under age 15 and in young adults up to age 34. Brain tumors are the second fastest growing cause of cancer death among those over age 65. There is an urgent need to have effective glioblastoma therapy to prolong these patients' lives and improve their quality of life.

Brain Tumor Grade Specification

Gliomas have been defined pathologically as tumors that display histological, immunohistochemical, and ultra-structural evidence of glial differentiation. The most widely used scheme for classification and grading of gliomas is that of the World Health Organization (WHO) (2). Gliomas are classified according to their hypothesized line of differentiation, that is, whether they display features of astrocytic, oligodendroglial, or ependymal cells. They are then graded on a scale of I to IV according to their degree of malignancy as judged by various histological features. Grade I tumors are biologically benign and can be surgically cured if deemed respectable at the time of diagnosis; grade II tumors are low-grade malignancies that may follow long clinical courses but are not curable by surgery; grade III tumors are malignant and lead to death within a few years; grade IV tumors (glioblastoma) are highly malignant, usually recalcitrant to chemotherapy, and lethal within 9-12 months (3).

Current Therapy for Brain Tumors

The major treatments consist of 1) Surgery, 2) Radiation therapy, 3) Chemotherapy, and 4) Biologic therapy. Since the brain poses a large problem for drug delivery, chemotherapy is usually co-delivered with a blood-barrier blocker (eg. mannitol). Over 50% of patients seek alternative therapies in addition to conventional treatment. Current chemotherapy in US include the following:

1) Anti-angiogenesis agents cuts off the blood supply of tumors. These agents currently or soon to be under investigation include thalidomide, TNP-470, platelet factor 4 (PF4), interferon and angiostatin.

2)Differentiating Agents are classes of drugs that can convert immature dividing tumor cells into mature cells, stopping tumor growth. Examples include retinoic acid, phenylacetate, and bryostatin.

3) Immunotherapy aims to make the immune system more effective in seeking out and destroying cancerous cells. Currently under investigation are several tools considered useful for boosting the immune system: Interferon, lymphocytes, and tumor vaccines.

4) Other treatments include drugs as follows: CPT-11, PCV, Tamoxifen, Thalidomide, VP-16/Etoposide, and BCNU. Adjuvant chemotherapy, usually with BCNU (1,3-bis (2-chlorethyl)-1-nitrosourea), increases survival slightly. Attempts to administer BCNU by arterial injection have been complicated by irreversible encephalopathy and ipsilateral visual loss owing to retinal toxicity.

Currently the most exciting chemotherapy drug for brain tumor is TEMODAR, which was approved by the US Food and Drug Administration (FDA) in August 1999 for adult patients with recurrent anaplastic astrocytoma. TEMODAR (temozolomide) is the first oral chemotherapeutic agent found to cross the blood-brain barrier. This oral, cytotoxic alkylating agent is the leader in a new class of compounds known as imidazotetrazines. The overall tumor response rate to TEMODAR was 22 percent, including complete responses (9 percent) and partial responses (13 percent). A complete response (CR) is defined as the loss of the tumor for at least two consecutive months as measured by MRI. A decrease of more than 50 percent in the tumor area for two months defined a partial response (PR).

Cisplatin, Taxol, 5FU Background

Cisplatin is a well-established cancer drug. Cisplatin was first synthesized in 1845, but its cytotoxic properties were not described until 1965. An experiment had been set up to see if an electric current would inhibit the reproduction of E. coli bacteria. The conclusion of the experiment was that electrolysis products from the platinum electrode were responsible for the inhibition. Cisplatin entered into clinical trials in 1971. Cisplatin is an inorganic complex formed by an atom of platinum surrounded by chloride and ammonia atoms in the cis position of a horizontal plane. Intracellularly, water displaces the chloride to form highly reactive charged platinum complexes. These complexes inhibit DNA through covalent binding leading to intrastrand, interstrand, and protein cross-linking of DNA. Experimental and clinical data suggest that cisplatin enhances radiation therapy effects. Early studies suggested that cisplatin was cell cycle phase-nonspecific, while more recent studies have shown complex and variable effects on the cell cycle.

Cisplatin's main uses are against bladder cancer, small cell lung cancer, ovarian cancer, and testicular cancer. Other cancers cisplatin can treatment include adrenocortical cancer, brain tumors, breast cancer, cervical cancer, endometrical cancer, gastrointestinal cancer, germ cell tumors, gynecological sarcoma, head and neck cancer, hepatoblastoma, malignant melanoma, neuroblastoma, non-hodgkin's lymphoma, osteosarcoma, and thyroid cancer.

Traxol and 5FU are both effective anti-cancer drugs. But they also induce MDR effects. Taxol is first discovered at the turn of last century, but the clinical trial of this drug started in 1983. Taxol is mainly used in breast cancer, ovarian cancer, head and neck cancer, and lung cancer. 5FU was developed in 1957 based on the observation that tumor cells utilized the base pair uracil for DNA synthesis more efficiently than did normal cells of the intestinal mucosa. It is a fluorinated pyrimidine that is metabolized intracellularly to its active form, fluodeoxyuridine monophophate (FdUMP). The active form inhibits DNA synthesis by ihibiting the normal production of thymidine. 5FU is cell cycle phase specific (S-phase). 5FU is mainly used in breast cancer, colorectal cancer, gastric cancer, and hepatic cancer. 5FU's less frequent uses include actinic keratosis, bladder cancer, cervical cancer, endometrial cancer, head and neck cancer, non-small cell lung cancer, ovarian cancer, pancreatic cancer, and prostate cancer.

MDR Effect of Cancer Cells

MDR effect of cancer cells is one major reason for the failure of many chemotherapeutic drugs. After cancer cells experience chemotherapeutic drug A, these cancer cells are not only resistant to drug A, but also resistant to drugs with different chemical structure, function, or inhibition mechanism from drug A. To date, overexpression of P170 glycoprotein on cell membrane is one of the main reasons causing MDR. P170 glycoprotein is a pump that is dependent on energy. P170 pumps out drugs from inside cells so that the cells could lower drug concentration inside cells—defined as MDR effect. So far scientists have discovered many MDR reversion drugs, summed up as follows: 1) calcium channel blockers, 2) calmodulin inhibitors, 3) Steroids and hormones, 4) immune modulators, 5) antibiotics. The above MDR reversion agents are effective in in vitro experiments, but are too toxic for human trials.

Cisplatin induces P-glycoprotein's expression. According to Yang et al's report, p-glycoprotein was expressed in ovarian cancer cell line following treatment with cisplatin (Yang, X, and Page, M, P-glycoprotein expression in ovarian cancer cell line following treatment with cisplatin, Oncol. Res. 1995, 7(12): 619-24). Human ovarian cancer cell line SKOV3 was grown during a period of four months in the presence of increasing concentrations of cisplatin (25-100 ng/ml). In the course of this treatment, the cells exhibited dramatic morphology changes, including reduction in cell size, loss of cellular projections and clustering. This was accompanied by the appearance of p-glycoprotein on the cell membrane. The new cell, designated SKOV3/CIS, acquired resistance to classical MDR drugs, such as doxorubicin, taxol, and actinomycine D. Verapamil enhanced the sensitivity of SKOV3/CIS to doxorubicin (260-fold), in conformity with the proposed mechanism of p-glycoprotein in MDR, but it did not potentate cisplatin cytotoxicity in SKOV3/CIS cells.

Certain drugs have been shown to reduce Cisplatin's MDR effect. In literature, SDZ PSC 833, a semisynthetic undecapeptide derived from cyclosporine D, is one of the most potent known inhibitors of the multidrug transporter P-glycoprotein (Baekelandt, M et al., Phase I/II trial of cisplatin and doxorubicin with SDZ PSC 833 in patients with refractory ovarian cancer, Proc. Annu. Meet. Am. Soc. Clin. Oncol 1997; 16: A757). Patients with histologically verified ovarian cancer were eligible if they had clinically resistant disease, defined as either stable disease after at least 3 cycles or disease progression after at least 2 cycles while treated with a combination of cisplatin and an anthracyclin. Treatment was then continued with Cisplatin 50 mg/m2 and doxorubicin with the addition of PSC. The maximal tolerated dose for doxonibicin was determined to be 35 mg/m2 with PSC. By administering SDZ PSC 833 intravenously together with cisplatin and doxorubicin, the clinicians observe major responded in heavily pretreated patients with progress disease, and acceptable toxicity.

Beta-elemene could reverse the MDR effect for cisplatin. This is the first report on beta-elemene's MDR reversion effect. The application of MDR-reversing agents is a potential principle means that conquers clinical drug resistance and improves the effect of chemotherapy. For nearly two decades, although many reversing compounds have been identified, clinical application of these agents is confined for their toxic and side effects.

Beta-elemene (2% emulsion), extracted from traditional medicine rhizoma zedoariae is a kind of non-cytotoxic anticancer drug. Clinical trials have demonstrated that beta-elemene emulsion exhibits no detriment to heart, liver, or kidney, and no inhibitory effect on bone marrow.

### SUMMARY OF THE INVENTION

The inventors propose a combination of cisplatin and beta-elemene, or a combination of cisplatin and beta-elemene's derivatives, or a combination of cisplatin and beta-elemene's intermediates in its chemical synthesis for the treatment of cancer, especially for the treatment of brain tumor, lung cancer, ovarian cancer, bladder cancer, cervical cancer, colon cancer, breast cancer, and prostate cancer. Beta-elemene and its related are effective not only in reversing multi-drug resistance in cancer cells, both in vitro and in vivo, but have been determined to be active as collateral sensitive agents, which are more cytotoxic towards MDR cells than normal cells, and as synergistic agents, which are more active in combination with other cytotoxic agents, such as cisplatin, than the individual drugs would be alone at the same concentrations. Beta-elemene or its related could lower cisplatin's IC50 to inhibit tumor grown in the following cell lines, brain tumor, lung cancer, ovarian cancer, bladder cancer, cervical cancer, colon cancer, breast cancer, and prostate cancer cell lines. Beta-elemene and its related will lower cisplatin's intake in cancer patients, and thus lowering cisplatin's side effects.

1) One object of the present invention is to provide processes for the preparation of beta-elemene and its derivatives useful as anticancer therapeutics.

2) Another object of the present invention is to provide various compounds useful as intermediates in the preparation of beta-elemene as well as analogues thereof.

3) A further object of the present invention is to provide synthetic methods for preparing such intermediates.

4) An additional object of the invention is to provide compositions useful in the treatment of subjects suffering from cancer comprising any of the analogues of the beta-elemene available through the preparative methods of the invention optionally in combination with pharmaceutical carriers.

5) A further object of the invention is to provide methods of treating subjects suffering from cancer using any of the analogues of beta-elemene available through the preparative methods of the invention optionally in combination with pharmaceutical carriers.

6) Another object of the invention is to use elemene and its related in a combination therapy against different cancer types with cisplatin, or Taxol, or 5FU. Beta-elemene and its related are effective not only in reversing multi-drug resistance in cancer cells, both in vitro and in vivo, but have been determined to be active as collateral sensitive agents, which are more cytotoxic towards MDR cells than normal cells, and as synergistic agents, which are more active in combination with other cytotoxic agents, such as cisplatin, than the individual drugs would be alone at the same concentrations. Beta-elemene or its related could lower cisplatin or Taxol, or 5FU) IC50 to inhibit tumor grown in the above cell lines, and they might lower cisplatin's intake in cancer patients, and thus lowering these cytotoxic drug's side effects.

### BRIEF DESCRIPTION OF THE DRAWINGS

FIG. 1 Two different synthetic schemes of (−)-beta-elemene.

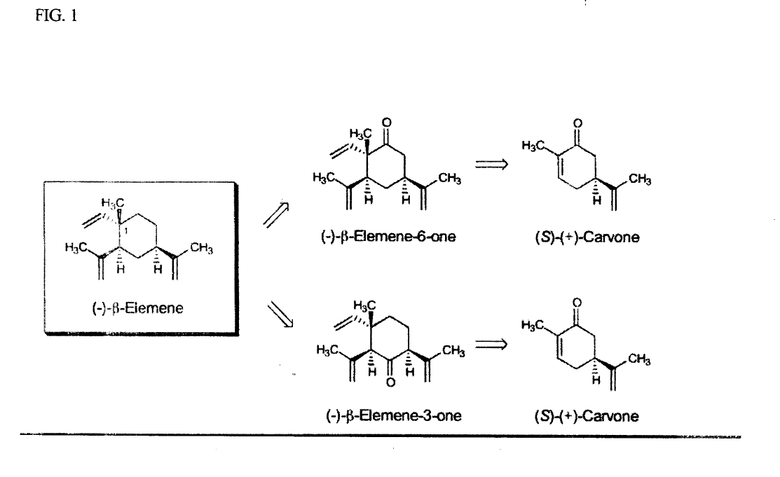

FIG. 2 Claims of elemene-like structures or derivatives.

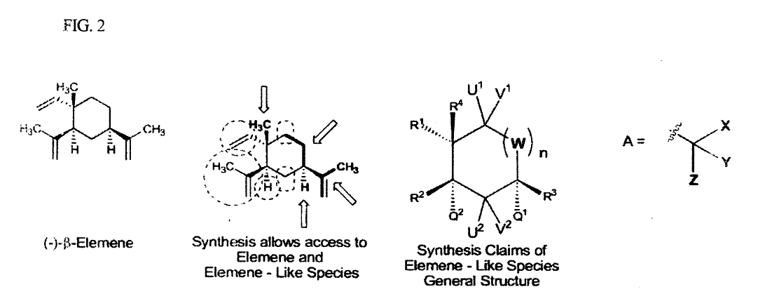

FIG. 3 Detailed description of two de novo synthesis routes of (−)-beta-elemene from (S)-(+)-Carvone.

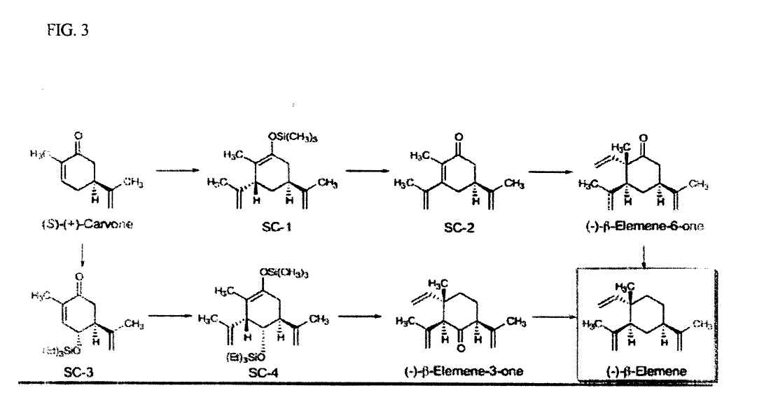

FIG. 4 Corey Synthesis analysis for (−)-beta-elemene.

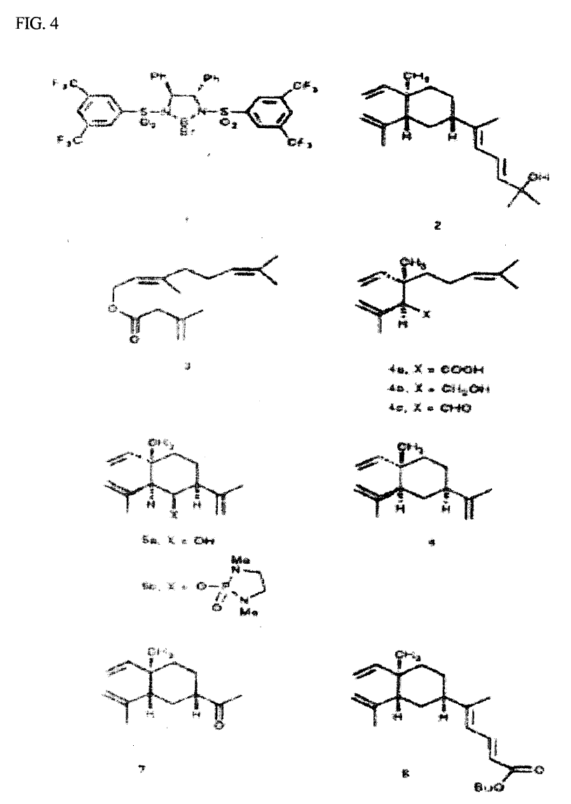

FIG. 5 Preparation of elemene derivative (+)-Fuscol from (R)-(−)-Carvone.

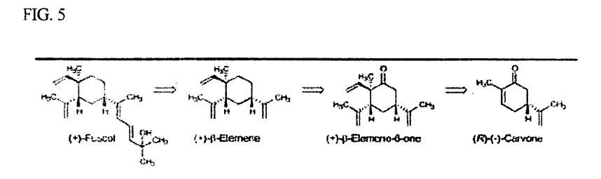

FIG. 6 Structures of ten (−)-beta-elemene derivatives synthesized.

### DETAILED DESCRIPTION OF THE INVENTION

1) Synthesis Route and Composition Claims

The inventors claimed the discovery of the unexpectedly efficacious, safe, non-multi drug resistant effect, nontoxic, and broadly applicable use of (−)-beta-Elemene as an anti-viral, anti-microbial, anti-biotic and especially as an anti-cancer chemotherapeutic; moreover, (−)-beta-Elemene derivatives and (−)-beta-Elemene-like structures are claimed, as are the processes by which said structures are obtained as well as the processes by which (−)-beta-Elemene is obtained. The use of (−)-beta-Elemene and (−)-beta-Elemene derivatives and (−)-beta-Elemene-like structures formulated singularly or in combination for anti-viral, anti-microbial, and anti-cancer applications is also claimed.

Synthesis of (−)-beta-Elemene

It is of interest to note that (−)-beta-Elemene has not been synthesized in enantiomerically pure form. Enantiomeric purity is critical for proper evaluation of a drug. For example, Thalidimide enantiomers are either highly effective medicines or horribly disfiguring teratogens, depending on the enantiomer. Given the major impact that our recent clinical studies of (−)-beta-Elemene formulated alone and in conjugation suggest, the inventors claim the synthesis of (−)-beta-Elemene and (−)-beta-Elemene-like molecules. Four synthetic plans are presented below.

Part 1: First, two de novo syntheses of (−)-beta-elemene and a wide range of (−)-beta-elemene-like compounds from (S)-(+)-Carvone is claimed. It is anticipated that:

A) Beginning with (S)-(+)-Carvone, (−)-β-Elemene derivative SC-1 can be readily procured by conjugate addition with a 2-propenyl unit, for example, via lithium di-2-propenyl cuprate (a Gilman reagent), and trapping of the enolate, for example with triethylsilyl chloride, to give the silyl enol ether. Conversion of SC-1 to SC-2 enables the formation of (−)-β-Elemene-6-one is in a short sequence as follows: Oxidation of enol ether SC-1 to enone SC-2 [using palladium (II)]. Subsequent 1,4-conjugate addition with hydride, for example effected with a copper reagent, followed by trapping with methyl iodide creates the α,α-dimethyl ketone. C—H bond activation of the equatorial methyl (using, for example, the oxime derived from the ketone) can be followed by further oxidation of the resultant alcohol to the aldehyde followed by olefination giving (−)-β-Elemene-6-one. The oxidant in C—H bond activation may be, for example, palladium (0) or platinum (II). Conversion of (−)-β-Elemene-6-one to (−)-β-Elemene, can be achieved by reduction (for example, hydrazine, potassium hydroxide, heat—a Wolff-Kishner reduction).

B) A second route using (S)-(+)-Carvone is oulined as well and is similar to Plan A above, however, this second route provides access to several other (−)-beta-Elemene-like molecules: Selective oxidation of (S)-(+)-Carvone at position 3 [using the (−)-beta-Elemene numbering], followed by suitable protection, if necessary, will give SC-3 (in the instance shown, protection of the 3-hydroxyl is given as the triethyl silyl ether). Following a similar course as in (A) above, SC-4 can be readily procured by conjugate addition with a 2-propenyl unit, for example, via the lithium di-2-propenyl cuprate (Gilman reagent) and trapping of the enolate as an enol ether (for example, with triethyl silyl chloride) as shown. Conversion of this adduct to (−)-beta-Elemene-3-one is outlined as follows: Oxidation of SC-4 to the enone can be achieved for example using palladium (II), followed by subsequent 1,4-conjugate addition of hydride (for example, effected with a copper reagent) followed by trapping with methyl iodide creates the a,a-dimethyl ketone. C—H bond activation of the equatorial methyl utilizing the oxime, derived from he ketone, followed by oxidation to the aldehyde and subsequent olefination of said aldehyde. The remaining carbonyl can be removed by reduction. Removal of the triethyl silyl ether to give the alcohol followed by oxidation will give (−)-beta-Elemene-3-one. Conversion of (−)-beta-Elemene-3-one to (−)-beta-Elemene can be achieved readily by reduction of the carbonyl.

Part 2: Based on Corey Synthesis

(+)-fuscol (##STR2##) of >99% pure via the intermediate terpenoid (−)-beta-elemene (##STR6##).

The reaction of geraniol with 1.1 equivalent of β, β-dimethylacryloyl chloride and 1.5 equivalent of triethylamine (CH2Cl2, −78 C, 3 h) afforded the β, γ-unsaturated ester ##STR3## (99% yield) in an interesting reaction that probably proceeds via a vinylketene intermediate. Treatment of ##STR3## in toluene with 1.1 equivalent of (S,S)-bromoborane ##STR1## and 8.3 equivalent of triethylamine (−70 C for 27 h, then 4 C for 36 h) afforded the Ireland-Claisen product ##STR4a## as a major product along with a minor diastereomer (85% total yield). Reduction of the mixture to the corresponding primary alcohols (LiAlH4, Et2O, 23 C, 24 h) and chromatography on AgNO3-impregnated silica gel gave diastereomerically pure ##STR4b## (70% yield) of >99% enantiomeric purity. Treatment of ##STR4c## with 1.1 equivalent of Et2AlCl (CH2Cl2, −78 C, 1.5 h) followed by extractive isolation and chromatography on silica gel-AgNO3 furnished the cyclized equatorial alcohol ##STR5a## (88% yield) along with 3% yield of less polar diastereomer (having equatorial hydroxyl and axial beta-isopropenyl substituents). Reaction of ##STR5a## with 2-chlorol-1,3-dimethyl-1,3,2-diazaphospholane and triethylamine (CH2Cl2, 23 C, 75 min) provided, after oxidation with 1.2 equivalent of H2O2 for 10 min, ##STR5b##, which was reduced with excess lithium and tert-amyl alcohol (4 equivalent) in liquid NH3-THF (−33 C, 10 h) to give (+)-beta-elemene (##STR6##, 95% yield), [alpha]23D+15.4 (c=0.6, CHCl3), which was indistinguishable, by NMR and infrared spectroscopic comparison, from an authentic sample of naturally derived (−)-beta-elemene.

(−)-beta-elemene (##STR6##) was converted to the methyl ketone ##STR7## by a two-step sequence. Catalytic dihydroxylation with the Sharpless phthalazine-linked bisether with dihydroquinidine, (DHQD)2-PHAL (0.1 equivalent), K2OsO4 (0.01 equivalent), K3Fe(CN)6 (3 equivalent), K2CO3 (3 equivalent), and CH3SO3NH2 (1 equivalent) in 1:1 tert-butyl alcohol-water at 0 C for 11 h afforded, after chromatography on silica gel, the diol resulting from selective attack at the isopropenyl appendage (1,4-) to the angular methyl group (76% yield; 92% yield corrected for recovered ##STR6##). Cleavage of the resulting 1,2-doil with 3 equivalent of NaIO4 (4:1 THF-H2O, 23 C, 30 min) gave ##STR7## in 96% yield. The highly selective attack of just one of the three double bonds of ##STR6## by OsO4 under catalysis by (DHQD)2-PHAL was predicted on the basis of the mechanistic model recently proposed for the asymmetric dihydroxylation reaction. Coupling the methyl ketone ##STR7## with 20 equivalent each of (n-BuO)2POCH2CH═CHCOOn-Bu and LiOt-Bu (added in four portions, THF solution, 23 C, 48 h) furnished the tetraene ester ##STR8## in 80% yield after chromatography on silica gel. Reaction of ##STR8## with 5 equivalent of MeLi (Et2O, −30 C, 12 h) afforded (+)-fuscol (##STR2##), [a]²³_{D}+19.7° (c=1, CHCl3), as a colorless oil in 95% yield.

As used herein, the term “linear or branched chain alkyl” encompasses, but is not limited to, methyl, ethyl, propyl, isopropyl, t-butyl, sec-butyl, cyclopentyl or cyclohexyl. The alkyl group may contain one carbon atom or as many as fourteen carbon atoms, but preferably contains one carbon atom or as many as nine carbon atoms, and may be substituted by various groups, which include, but are not limited to, acyl, aryl, alkoxy, aryloxy, carboxy, hydroxy, carboxamido and/or N-acylamino moieties.

As used herein, the terms “alkoxycarbonyl”, “acyl” and “alkoxy” encompass, but are not limited to, methoxycarbonyl, ethoxycarbonyl, propoxycarbonyl, n-butoxycarbonyl, benzyloxycarbonyl, hydroxypropylcarbonyl, aminoethoxycarbonyl, sec-butoxycarbonyl and cyclopentyloxycarbonyl. Examples of acyl groups include, but are not limited to, formyl, acetyl, propionyl, butyryl and penanoyl. Examples of alkoxy groups include, but are not limited to, methoxy, ethoxy, propoxy, n-butoxy, sec-butoxy and cyclopentyloxy.

As used herein, an “aryl” encompasses, but is not limited to, a phenyl, pyridyl, pyrryl, indolyl, naphthyl, thiophenyl or furyl group, each of which may be substituted by various groups, which include, but are not limited, acyl, aryl alkoxy, aryloxy, carboxy, hydroxy, carboxamido or Nacylamino moieties. Examples of aryloxy groups include, but are not limited to, a phenoxy, 2-methylphenoxy, 3-methylphenoxy and 2-naphthoxy. Examples of acyloxy groups include, but are not limited to, acetoxy, propanoyloxy, butyryloxy, pentanoyloxy and hexanoyloxy.

The subject invention provides chemotherapeutic analogues of beta-elemene, including a compound having the structure: ##STR7## and ##STR8##.

wherein R, R.sub.0, and R′ are independently H, linear or branched chain alkyl, optionally substituted by hydroxy, alkoxy, fluorine, NR.sub.1 R.sub.2, N-hydroximino, or N-alkoxyimino, wherein R.sub.1 and R.sub.2 are independently H, phenyl, benzyl, linear or branched chain alkyl; wherein R″ is—CHY.dbd.CHX, or H, linear or branched chain alkyl, phenyl, 2-methyl-1,3-thiazolinyl, 2-furanyl, 3-furanyl, 4-furanyl, 2-pyridyl, 3-pyridyl, 4-pyridyl, imidazolyl, 2-methyl-1,3-oxazolinyl, 3-indolyl or 6-indolyl; and wherein X is H, linear or branched chain alkyl, phenyl, 2-methyl-1,3-thiazolinyl, 2-furanyl, 3-furanyl, 4-furanyl, 2-pyridyl, 3-pyridyl, 4pyridyl, imidazolyl, 2-methyl-1,3-oxazolinyl, 3-indolyl or 6-indolyl; wherein Y is H or linear or branched chain alkyl; wherein Z is O, N(OR.sub.3) or N—NR.sub.4 R.sub.5, wherein R.sub.3, R.sub.4 and R.sub.5 are independently H or a linear or branched alkyl; and wherein n is 0, 1, 2, or 3. In one embodiment, the invention provides the compound having the structure: ##STR6##

wherein R is H, methyl, ethyl, n-propyl, n-butyl, n-hexyl, CH.sub.2 OH, or (CH.sub.2).sub.3 OH.

The invention also provides a compound having the structure: ##STR4##

wherein R, R.sub.0, and R′ are independently H, linear or branched chain alkyl, optionally substituted by hydroxy, alkoxy, fluorine, NR.sub.1 R.sub.2, N-hydroximino, or N-alkoxyimino, wherein R.sub.1 and R.sub.2 are independently H, phenyl, benzyl, linear or branched chain alkyl; wherein R″ is —CHY.dbd.CHX, or H, linear or branched chain alkyl, phenyl, 2-methyl-1,3-thiazolinyl, 2-furanyl, 3-furanyl, 4-furanyl, 2-pyridyl, 3-pyridyl, 4-pyridyl, imidazolyl, 2-methyl-1,3-oxazolinyl, 3-indolyl or 6-indolyl; and wherein X is H, linear or branched chain alkyl, phenyl, 2-methyl-1,3-thiazolinyl, 2-furanyl, 3-furanyl, 4-furanyl, 2-pyridyl, 3-pyridyl, 4pyridyl, imidazolyl, 2-methyl-1,3-oxazolinyl, 3-indolyl or 6-indolyl; wherein Y is H or linear or branched chain alkyl; wherein Z is O, N(OR.sub.3) or N—NR.sub.4 R.sub.5, wherein R.sub.3, R.sub.4 and R.sub.5 are independently H or a linear or branched chain alkyl; and wherein n is 0, 1, 2, or 3. In a certain embodiment, the invention provides a compound having the structure: ##STR4##

wherein R is H, methyl, ethyl, n-propyl, n-butyl, n-hexyl, or CH.sub.2 OH.

In addition, the invention provides a compound having the structure: ##STR5##

wherein R, R.sub.0, and R′ are independently H, linear or branched chain alkyl, optionally substituted by hydroxy, alkoxy, fluorine, NR.sub.1 R.sub.2, N-hydroximino, or N-alkoxyimino, wherein R.sub.1 and R.sub.2 are independently H, phenyl, benzyl, linear or branched chain alkyl; wherein R″ is —CHY.dbd.CHX, or H, linear or branched chain alkyl, phenyl, 2-methyl-1,3-thiazolinyl, 2-furanyl, 3-furanyl, 4-furanyl, 2-pyridyl, 3-pyridyl, 4-pyridyl, imidazolyl, 2-methyl-1,3-oxazolinyl, 3-indolyl or 6-indolyl; and wherein X is H, linear or branched chain alkyl, phenyl, 2-methyl-1,3-thiazolinyl, 2-furanyl, 3-furanyl, 4-furanyl, 2-pyridyl, 3-pyridyl, 4pyridyl, imidazolyl, 2-methyl-1,3-oxazolinyl, 3-indolyl or 6-indolyl; wherein Y is H or linear or branched chain alkyl; wherein Z is O, N(OR.sub.3) or N—NR.sub.4 R.sub.5, wherein R.sub.3, R.sub.4 and R.sub.5 are independently H or a linear or branched chain alkyl; and wherein n is 0, 1, 2, or 3. In particular, the invention provides a compound having the structure: ##STR6##

wherein R is H, methyl, ethyl, n-propyl, n-butyl, CH.sub.2 OH or (CH.sub.2).sub.3 OH.

The invention further provides a compound having the structure: ##STR7##

wherein R, R.sub.0 and R′ are independently H, linear or branched chain alkyl, optionally substituted by hydroxy, alkoxy, fluorine, NR.sub.1 R.sub.2, N-hydroximino or N-allcoxyimino, wherein R.sub.1 and R.sub.2 are independently H, phenyl, benzyl, linear or branched chain alkyl; wherein R″ is —CHY.dbd.CHX, or H, linear or branched chain alkyl, phenyl, 2-methyl-1, 3-thiazolinyl, 2-furanyl, 3-furanyl, 4-furanyl, 2-pyridyl, 3-pyridyl, 4-pyridyl, imidazolyl, 2-methyl-1,3-oxazolinyl, 3-indolyl or 6-indolyl; and wherein X is H, linear or branched chain alkyl, phenyl, 2-methyl-1,3-thiazolinyl, 2-furanyl, 3-furanyl, 4-furanyl, 2-pyridyl, 3-pyridyl, 4-pyridyl, imidazolyl, 2-methyl-1,3-oxazolinyl, 3-indolyl or 6-indolyl; wherein Y is H or linear or branched chain alkyl; wherein Z is O, N(OR.sub.3) or N—NR.sub.4 R.sub.5, wherein R.sub.3, R.sub.4 and R.sub.5 are independently H or a linear or branched chain alkyl; and wherein n is 0, 1, 2 or 3.

Advantages Over Prior Part on Synthesis Route in Part 1

In addition to being the only enantioselective synthesis of (−)-b-Elemene, this route is stereoselective and general with respect to modification of the scaffold. Unlike other syntheses, this route provides access to C1, C2, C3, C4, C5, and C6 derivatives, including the removal of the isopropenyl group at C4, and derivitization of the methyl group of C1.

Each of These is Outlined Below:

C1. The C1 position can be manipulated selectively in the 1,4-conjugate addition step delivering hydride to position C2 followed by alkylation. The alkylating group could be widely varied and in such case responds to R4 of the general structure shown in the Scheme. If the alkylating agent is methyl such that the a,a-ketone is produced, subsequent oxidation of the equatorial methyl corresponding to group R1 can be achieved, furthermore, manipulation of this oxidized methyl as an alcohol, a ketone, or other carbonyl derivative, as well as subsequent derivitization of such carbonyl derivatives giving rise to a wide range of R1 substituents can be readily achieved. Hence both R1 and R4 can be manipulated at will with this synthesis, both of these being on position C1.

C2. The C2 position can be manipulated selectively as well. Group R2 and Q2 on position C2 is selectively added in either of two ways. First, using synthesis route A: 1,4-conjugate addition producing structures like SC-1 and, subsequently, SC-2, installs these groups. A wide range of substituents can be introduced and manipulated in this way. This versatility is present in following path B as well; however, path B has additional versatility. (−)-b-Elemene-3-one can, in principle, be derivitized selectively at the C2 position, depending on adjacent substituents on position C4, taking advantage of carbonyl/enolate reactivity. C3. Position C3 can be selectively derivitized using path B, for example, SC-3 and SC-4 and (−)-b-Elemene-3-one each represent modification on the C3 position; moreover, replacement of the triethyl silyloxy group of SC-3 or SC-4 or derivitization of the ketone on C3 of (−)-b-Elemene-3-one can be achieved selectively and replaced with a wide range of substituents as U2 and V2.

C4. Position C4 derivatives can be obtained readily as well. It is important to note that there is an inherent near-symmetry of SC-1 and SC-4 and this near-symmetry allows for direct access to (−)-b-Elemene-like compounds. In addition, both path A and B allows direct control over substituents at C4. For example, oxidation of the 2-propenyl group at C4 (this can be achieved directly on carvone) generates (−)-b-Elemene-6-one-like and (−)-b-Elemene-3-one-like derivatives that can be substituted at the C4 position readily (introducing group Q1). Indeed, removal of the 2-propenyl group at C4 can be achieved by oxidation of the olefin to the ketone followed by retro-Claisen condensation. Derivitization of this isopropenyl unit is also readily achieved. Thus, a wide range of Q1 and R3 groups can be introduced selectively at C4.

C5. Following standard protocols, a,a-disubstituted ketones, for example, a,a-dimethyl ketone and other compositions related to (−)-b-Elemene-6-one, can be selectively derivitized in the C5 position taking recourse to enolate chemistry and giving rise to U3 and V3 substituents.

C6. Modifications at C6 can be achieved in a manner analogous to modifications at C3, i.e. carbonyl derivatives can readily be prepared stereoselectively and further modification, for example, olefination, as well as other substituents can be added including a wide range of E1 and V1 substituents.

In addition to the changes outlined, it should be noted that ring expansion and ring contraction can also be achieved to give rise to (−)-b-Elemene derivatives containing either five or seven atoms in the central ring. The identity of W can be a carbon, nitrogen, or oxygen, and can also, in the case of carbon bearing substituents equivalent to U and V identity. Similarly, if W is nitrogen the group R can be widely varied to include a wide range of substituents as outlined below.

Derivatives of (−)-beta-elemene synthesized and tested for tumor cell line growth inhibition

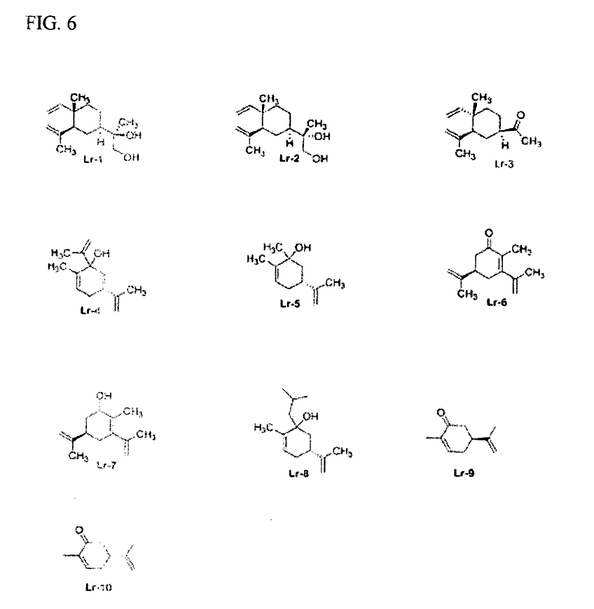

Ten derivatives of (−)-beta-elemene (FIG. 6) is synthesized and tested for in vitro tumor cell line inhibition.

2) Anti-tumor Usage Claims

In addition, the invention provides a method of treating cancer in a subject suffering therefrom comprising administering to the subject a therapeutically effective amount of any of the analogues related to (−)-beta-elemene disclosed herein optionally in combination with a pharmaceutically suitable carrier. The method may be applied where the cancer is a solid tumor or leukemia. In particular, the method is applicable where The cancer is brain tumor, lung cancer and colorectal cancer.

The subject invention also provides a pharmaceutical composition for treating cancer comprising any of the analogues of (−)-beta-elemene disclosed hereinabove, as an active ingredient, optionally though typically in combination with a pharmaceutically suitable carrier. The pharmaceutical compositions of the present invention may further comprise other therapeutically active ingredients.

The subject invention further provides a method of treating cancer in a subject suffering therefrom comprising administering to the subject a therapeutically effective amount of any of the derivatives of (−)-beta-elemene disclosed herein above and a pharmaceutically suitable carrier. The method is especially useful where the cancer is a solid tumor or leukemia.

The compounds taught above which are related to (−)-beta-elemene are useful in the treatment of cancer, and particularly, in cases where multidrug resistance is present, both in vivo and in vitro. The ability of these compounds as nonsubstrates of MDR in cells, as demonstrated in the Tables below, shows that the compounds are useful to treat, prevent or ameliorate cancer in subjects suffering therefrom.

The magnitude of the therapeutic dose of the compounds of the invention will vary with the nature and severity of the condition to be treated and with the particular compound and its route of administration. In general, the daily dose range for anticancer activity lies in the range of 3-300 mg/kg of body weight in a mammal, preferably 10-40 mg/kg, and most preferably 10-20 mg/kg, in single or multiple doses. In unusual cases, it may be necessary to administer doses above 80 mg/kg.

Any suitable route of administration may be employed for providing a mammal, especially a human, with an effective dosage of a compound disclosed herein. For example, oral, rectal, topical, parenteral, ocular, pulmonary, nasal, etc., routes may be employed. Dosage forms include tablets, troches, dispersions, suspensions, solutions, capsules, creams, ointments, aerosols, etc.

The compositions include compositions suitable for oral, rectal, topical (including transdermal devices, aerosols, creams, ointments, lotions and dusting powders), parenteral (including subcutaneous, intramuscular, intraarterial, and intravenous), ocular (ophthalmic), pulmonary (nasal or buccal inhalation) or nasal administration. Although the most suitable route in any given case will depend largely on the nature and severity of the condition being treated and on the nature of the active ingredient. They may be conveniently presented in unit dosage form and prepared by any of the methods well known in the art of pharmacy.

In preparing oral dosage forms, any of the unusual pharmaceutical media may be used, such as water, glycols, oils, alcohols, flavoring agents, preservatives, coloring agents, and the like in the case of oral liquid preparations (e.g., suspensions, elixers and solutions); or carriers such as starches, sugars, microcrystalline cellulose, diluents, granulating agents, lubricants, binders, disintegrating agents, etc., in the case of oral solid preparations are preferred over liquid oral preparations such as powders, capsules and tablets. If desired, capsules may be coated by standard aqueous or non-aqueous techniques. In addition to the dosage forms described above, the compounds of the invention may be administered by controlled release means and devices.

Pharmaceutical compositions of the present invention suitable for oral administration may be prepared as discrete units such as capsules, cachets or tablets each containing a predetermined amount of the active ingredient in powder or granular form or as a solution or suspension in an aqueous or nonaqueous liquid or in an oil-in-water or water-in-oil emulsion. Such compositions may be prepared by any of the methods known in the art of pharmacy. In general compositions are prepared by uniformly and intimately admixing the active ingredient with liquid carriers, finely divided solid carriers, or both and then, if necessary, shaping the product into the desired form. For example, a tablet may be prepared by compression or molding, optionally with one or more accessory ingredients. Compressed tablets may be prepared by compressing in a suitable machine the active ingredient in a free-flowing form such as powder or granule optionally mixed with a binder, lubricant, inert diluent or surface active or dispersing agent. Molded tablets may be made by molding in a suitable machine, a mixture of the powdered compound moistened with an inert liquid diluent.

The present invention will be better understood from the Experimental Details which follow. However, one skilled in the art will readily appreciate that the specific methods and results discussed are merely illustrative of the invention as described in the claims which follow thereafter. It will be understood that the processes of the present invention for preparing beta-elemene, derivatives thereof and intermediates thereto encompass the use of various alternate protecting groups known in the art. Those protecting groups used in the disclosure including the Examples below are merely illustrative.

3) Combination Therapy for Cancer Treatment

The preferred mode of invention without limiting its use or use of pharmaceutical equivalents to those described herein is to administer a therapeutic dose of a cisplatin, in combination with a therapeutic dose of beta-elemene or its related starting with the minimum recommended doses of each drug.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. A therapeutic change is a change in a measured biochemical characteristic in a direction expected to alleviate the disease or condition being addressed. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. The term “therapeutic window” is intended to mean the range of dose between the minimal amount to achieve any therapeutic change, and the maximum amount, which results in a response that is the response immediately before toxicity to the patient.

The dosage regimen utilizing cisplatin or taxol, or 5FU in combination with beta-elemene and its related is selected in accordance with a variety of factors including type, species, age, weight, sex and medical condition of the patient; the severity of the condition to be treated; the route of administration; the cardiac, renal and hepatic function of the patient; and the particular compound or salt or ester thereof employed. Dosages in all events should be limited to the therapeutic window. Since two different active agents are being used together in a combination therapy, the potency of each of the agents and the interactive effects achieved by combining them together must also be taken into account. A consideration of these factors is well within the purview of the ordinarily skilled clinician for the purpose of determining the therapeutically effective or prophylactically effective amount.

The subject invention also provides a pharmaceutical composition for treating cancer comprising cisplatin or Taxol or 5FU and any of the analogues of beta-elemene, as an active ingredient, optionally though typically in combination with a pharmaceutically suitable carrier. The pharmaceutical compositions of the present invention may further comprise other therapeutically active ingredients.

The subject invention further provides a method of treating cancer in a subject suffering wherefrom comprising administering to the subject a therapeutically effective amount of cisplatin or Taxol or 5FU and any of beta-elemene and its related and a pharmaceutically suitable carrier. The method is especially useful where the cancer is a solid tumor, such as brain tumor, lung cancer, ovarian cancer, bladder cancer, cervical cancer, colon cancer, breast cancer, and prostate cancer.

Beta-elemene and its analogs are useful in the treatment of cancer, and particularly, in cases where multidrug resistance is present, both in vivo and in vitro. The ability of these compounds as non-substrates of MDR in cells, as demonstrated in the Tables below, shows that the compounds are useful to treat, prevent or ameliorate cancer in subjects suffering therefrom.

Any suitable route of administration may be employed for providing a mammal, especially a human, with an effective dosage of a compound disclosed herein. For example, oral, rectal, topical, parenteral, ocular, pulmonary, nasal, etc., routes may be employed. Dosage forms include tablets, troches, dispersions, suspensions, solutions, capsules, creams, ointments, aerosols, etc.

The compositions include compositions suitable for oral, rectal, topical (including transdermal devices, aerosols, creams, ointments, lotions and dusting powders), parenteral (including subcutaneous, intramuscular and intravenous), ocular (ophthalmic), pulmonary (nasal or buccal inhalation) or nasal administration. Although the most suitable route in any given case will depend largely on the nature and severity of the condition being treated and on the nature of the active ingredient. They may be conveniently presented in unit dosage form and prepared by any of the methods well known in the art of pharmacy.

In preparing oral dosage forms, any of the unusual pharmaceutical media may be used, such as water, glycols, oils, alcohols, flavoring agents, preservatives, coloring agents, and the like in the case of oral liquid preparations (e.g., suspensions, elixers and solutions); or carriers such as starches, sugars, microcrystalline cellulose, diluents, granulating agents, lubricants, binders, disintegrating agents, etc., in the case of oral solid preparations are preferred over liquid oral preparations such as powders, capsules and tablets. If desired, capsules may be coated by standard aqueous or non-aqueous techniques. In addition to the dosage forms described above, the compounds of the invention may be administered by controlled release means and devices.

Phannaceutical compositions of the present invention suitable for oral administration may be prepared as discrete units such as capsules, cachets or tablets each containing a predetermined amount of the active ingredient in powder or granular form or as a solution or suspension in an aqueous or nonaqueous liquid or in an oil-in-water or water-in-oil emulsion. Such compositions may be prepared by any of the methods known in the art of pharmacy. In general compositions are prepared by uniformly and intimately admixing the active ingredient with liquid carriers, finely divided solid carriers, or both and then, if necessary, shaping the product into the desired form. For example, a tablet may be prepared by compression or molding, optionally with one or more accessory ingredients. Compressed tablets may be prepared by compressing in a suitable machine the active ingredient in a free-flowing form such as powder or granule optionally mixed with a binder, lubricant, inert diluent or surface active or dispersing agent. Molded tablets may be made by molding in a suitable machine, a mixture of the powdered compound moistened with an inert liquid diluent.

### EXAMPLES

### Example 1

Synthesis of ##STR3##

(E)-Geranyl 3-Methyl-3-butenate

A solution of geraniol (225 ul, 1.29 mmol, 1.0 equivalent) and triethylamine (271 ul, 1.94 mmol, 1.5 equivalent) in dry dichloromethane (1 ml) was cooled to −78 C and treated dropwise with 3,3-dimethylacryloyl chloride (159 ul, 1.43 mmol, 1.1 equivalent). After 3 h, the solution was diluted with water (1 ml) and dichloromethane (1 ml), and the cooling hath was removed. The mixture was extracted with dichloromethane (3×20ml), and the combined organics were dried (MgSO4) and concentrated in vacuo. Purification by radial chromatography (4 mm SiO2 plate; elute, 7% EtOAc-hexanes; product, fractions 4-6; 30 ml/fraction) afforded ##STR3## (301 mg, 1.27 mmol, 99% yield) as a clear oil: Rf starting material, 0.14; product, 0.51 (5:1 hexanes-EtOAc, anisaldehyde); FTIR (film) 2970, 2919, 2858, 1738, 1653, 1445, 1377, 1206, 1153, 987, 896 cm-1; .sup. 1H NMR (400 MHz, CDCl3) δ5.31-5.35 (m, 1H), 5.04-5.08 (m, 1H), 4.88 (bs, 1H), 4.83 (bs, 1H), 4.60 (s, 1H), 4.58 (s, 1H), 3.01 (s, 2H), 2.00-2.09 (m, 4H), 1.79 (s, 3H), 1.69 (s, 3H), 1.66 (s, 3H), 3.01 (s, 2H), 2.00-2.09 (m, 4H), 1.79 (s, 3H), 1.69 (s, 3H), 1.66 (s, 3H), 1.58 (s, 3H); .sup.13 C NMR (101 Mhz, CDC13) δ171.2, 142.2, 138.6, 131.7, 123.7, 118.2, 114.5, 61.4, 43.4, 39.4, 26.2, 25.6, 22.3, 17.6, 16.4; HRMS (EI, Pos) m/z calculated for [Cl5H2402]+236.1776, found 236.1768.

### Example 2

Synthesis of ##STR4a##

(2S, 3S)-2-Isopropenyl-3,7-dimethyl-3-vinyl-6-octenoic Acid

The 3,5-bis(trifluoromethyl)benzenesulfonamide of (R,R)-1,2-diphenyl-1,2-diaminoethane (718 mg, 0.940 mmol, 1.0 equivalent) was dried under vacuum at 70 C for 3 h. The reaction flask was then evacuated and flushed three times with dry N2. Freshly distilled dichloromethane (32 ml) was added, and the homogeneous solution was cooled to −78 C. After 10 min, freshly distilled Bbr3 (3.76 ml, 0.5 M in CH2C12, 1.88 mmol, 2.0 equivalent) was added and the solution was stirred for 5 min at −78 C and then warmed to 23 C. After 16 h, all volatile materials were removed under vacuum, the resulting white solid was redissolved in dichloromethane (20 ml), and the solution was concentrated again. After 60 min, the flask was evacuated and flushed three times with N2, and the resultant white solid was dissolved in freshly distilled toluene (32 ml). The bromoborane complex (##STR1##) was cooled to −78 C, Et3N (983 ul, 7.05 mmol, 7.5 equivalent) was added dropwise, and the mixture was stirred to effect solution (25 min). A precooled solution of ##STR3## (175 mg, 0.740 mmol, 0.8 equivalent) in toluene (4 ml) was added dropwise at −78 C, and the resultant solution was stirred at −70 C for 27 h and subsequently warmed to 4 C. After 36 h, the reaction solution was warmed to 23 C, diluted with diethyl ether (40 ml), acidified to pH 1 with 10% HCl, and extracted with diethyl ether (4×60 ml). The ethereal extract was dried (MgSO4) and concentrated in vacuo to give a 3:1 mixture of ##STR4a## and a minor diastereomer as a yellow oil (149.2 mg, 0.631 mmol, 85% yield): Rf starting material, 0.71; product, 0.26 (5% MeOH-CHCl3, Verghns); FTIR (film) 3084, 3055, 2972, 2927, 2859, 2729, 1707, 1638, 1452, 1413, 1377, 1265, 916, 742 cm-1; .sup.1H NMR (400 MHz, CDCl3) δ6.09, 5.86 (dd, 1H, J=10.9, 17.5, major), 4.96-5.12 (m, 5HO, 3.08 (s, 1H, major), 3.07 (s, 1H, minor), 1.85-1.91 (m, 2H), 1.85 (s, 3H), 1.67 (s, 3H), 1.60 (s, 3H), 1.41-1.57 (m, 2H), 1.18 (s, 3H, major), 1.12 (s, 3H, minor); HRMS (EI, Pos) m/z calculated for [C 15H24O2]+236.1776, found 236.1783.

### Example 3

Synthesis of ##STR4b##

(2S, 3S)-2-Isopropenyl-3,7-dimethyl-3-vinyl-6-octenol A mixture of ##STR4a## and minor diastereomer (18 mg, 0.076 mmol, 1.0 equivalent) in dry diethyl ether (2 ml) was treated with LiAlH4 (15 mg, 0.381 mmol, 5.0 equivalent) at 23 C.

After 12 h, additional LiAlH4 (15 mg, 0.381 mmol, 5.0 equivalent) and diethyl ether (2 ml) were added. After an additional 12 h, H2O (50 ul), NaOH (15% w/v, 50 ul), and H2O (150 ul) were added sequentially. The mixture was stirred for 10 min, filtered, dried (MgSO4), and concentrated in vacuo. Flash chromatography (10 g of SiO2; eluent, 10% EtOAc-hexanes; product, fractions 7-21; 10 ml/fraction) yielded a 3:1 mixture of ##STR4b## and minor diastereomer as a clear oil (15.8 mg, 0.071 mmol, 93% yield): Rf starting material, 0.46; product, 0.72 (12% MeOH—CHC13, anisaldehyde). The 3:1 mixture of diastereomers was separated by AgNO3-impregnated radial chromatography (4 mm SiO2 plate; eluent, 4:1 EtOAc-hexanes; minor, fractions 11-15; ##STR4b##, fractions 16-35; 30 ml/fraction) followed by passage through silica gel (20 g; 200 ml of 10% EtOAc-hexanes) to afford diastereomerically pure ##STR4b##: AgNO3-impregnated TLC: Rf ##STR4b##, 0.20; minor 0.35 (12% MeOH—CHC13. anisaldehyde). The enantiomeric purity of ##STR4b## was determined to be greater than 99:1 by chiral high-performance liquid chromathography (Chiralcel OD colume, 1% 2-propanol-hexanes, 214 nm, 1 ml/min, retention times S,S-isomer, ##STR4b##=9.4 min, R,R-isomer=23 min): [α]²³_{D}−40.2° (c=0.54, CHCl3); FTIR (film) 3377, 3080, 2969, 2925, 2858, 1639, 1450, 1414, 1376, 1033, 1005, 912, 893 cm-1; .sup.1H NMR (500 MHz, CDCl3) δ5.80 (dd, 1H, J-10.8, 17.5), 5.02-5.08 (m, 3H), 4.91 (dd, 1H, J=1.3, 17.5), 4.83 (d, 1H, J=1.6), 3.72 (dd, 1H, J=4.3, 10.7), 1.82-1.90 (m, 2H), 1.77 (m, 3H), 1.67 (d, 3H, J=0.8), 1.57 (s, 3H), 1.30-1.44 (m, 2H), 1.04 (s, 3H); .sup.13 C NMR (101 MHz, CDCl3) d 144.4, 144.3, 131.3, 124.7, 115.7, 112.8, 61.1, 58.6, 41.2, 39.4, 25.7, 23.2, 22.6, 20.8, 17.6; HRMS (Cl, NH3) m/z calculated for [C15H26O]+NH3 240.2327, found 240.2317.

### Example 4

Synthesis of ##STR4c##

(2S,3S)-2-isopropenyl -3,7-dimethyl-3-vinyl-6-octenal

A suspension of Dess-martin reagent (232 mg, 0.546 mmol, 1.5 equivalent) in dry dichloromethane (5 ml) was added to ##STR4b## (81 mg, 0.364 mmol, 1.0 equivalent) in dichloromethane (2 ml) at 23 C. After 1 h, the solution was filtered through Celite 545, concentrated in vacuo, rediluted in hexanes, and filtered through Celite 545. The filtrate was concentrated in vacuo and purified by flash chromatography (10 g of SiO2;eluent, 4% EtOAc-hexanes, product, fractions 48; 10 ml/fraction) to afford ##STR4c## (79 mg, 0.359 mmol, 98% yield) as a clear oil; Rf starting material, 0.28; product, 0.58 (5:1 hexanes-EtOAc, anisaldehyde); [α]²³_{D}−40.20 (c=0.91, CHCl3); FTIR (film) 2970, 2921, 2859, 1721, 1638, 1453, 1377, 914 cm-1; .sup.1H NMR (500 MHz, CDCl3) δ9.65 (d, 1H, J=4.5), 5.92 (dd, 1H, J=10.9, 17.6), 5.14-5.17 (m, 2H), 5.06 (t, 1H, J=7.1), 5.00 (d, 1H, J=17.6), 4.88 (s, 1H), 2.70 (s, 3H), 1.38-1.50 (m, 2H), 1.15 (s, 3H), 1.67 (s, 3H), 1.57 (s, 3H), 1.38-1.50 (m, 2H), 1.15 (s, 3H); sup.13 C NMR (126 MHz, CDCl3) d 202.0, 143.1, 139.5, 131.5, 124.2, 116.8, 114.2, 67.1, 42.3, 39.1, 25.7, 25.6, 22.4, 20.6, 17.6; HRMS (EI, Pos) m/z calculated for [C15H24O]+220.1827, found 220.1817.

### Example 5

Synthesis of ##STRSa##

(1S,2S,3S,6S)-2,6-Diisopropenyl-3-methyl-3-vinylcyclohexanol

Diethylaluminum chloride (210 ul, 1.8 M in toluene, 0.379 mmol, 1.1 equivalent) was added dropwise to a solution of ##STR4c## (76 mg, 0.344 mmol, 1.0 equivalent) in dry dichloromethane (10 ml) at −78 C. Agter 1.5 h, triethylamine (500 ul) was added, the cooling bath was removed, and the solution was added to a mixture of saturated NaHCO3 (20 ml) and dichloromethane (2×20ml), and the organic fractions were combined, dried (MgSO4), and concentrated in vacuo. Flash chromatography (15 g of SiO2; eluent, 4% EtOAc-hexanes; product, fractions 11-23; 10 ml/fraction) afforded a 96:4 mixture of ##STR5a## and a minor diastereomer (70.1 mg, 0.318 mmol, 92% yield): Rf starting material, 0.58; product, 0.41 (5:1 hexanes-EtOAc, anisaldehyde). The diastereomeric mixture was separated by AgNO3-impregnated radial chromatography (2 mm plate; eluent, 5:1 EtOAc-hexanes; product, fractions 10-33; 3 ml/fraction) followed by passage through silica gel (10 g; 150 ml of 4% EtOAc-hexanes) to afford pure ##STR5a## (88% yield) as a clear oil: AgNO3-impregnated TLC: Rf ##STR5a##, 0.08; minor, 0.17 (12% MeOH—CHCl3; anisaldehyde); [α]²³_{D}+17.80 (c=0.91, CHCl3); FTIR (film) 3566, 3486, 2969, 2931, 1639, 1454, 1375, 1004, 910, 889 cm-1; sup.1H NMR (500 MHz, CDCl3) δ 5.78 (dd, 1H, J=10.9, 17.4), 5.06 (s, 1H), 4.88-4.92 (m, 4H), 4.76 (s, 1H), 3.77 (t, 1H, J=10.4), 2.08 (dt, 1H, J=4.8, 10.8), 1.98 (d, 1H, J=10.4), 1.90 (bs, 1H), 1.80 (s, 3H), 1.79 (s, 3H), 1.51-1.66 (m, 3H), 1.42 (dt, 1H, J=3.1, 13.0), 1.06 (s, 3H); sup.13 C NMR (101 MHz, CDC13) d 148.9, 147.1, 144.2, 114.1, 112.2, 110.3, 69.3, 59.7, 53.7, 41.3, 39.0, 26.2, 25.0, 19.5, 18.1; HRMS (EI, Pos) m/z calculated for [C15H24O]+220.1827, found 220.1826.

### Example 6

Synthesis of ##STR5b##

Reaction of 2-chloro-1,3-dimethyl-1,3,2-diazaphospholane with ##STR5a## to get ##STR5b##

2-chloro-1,3-dimethyl-1,3,2-diazaphospholane (10 ul, 0.076 mmol, 1.4 equivalent) was added dropwise to a solution of ##STR5a## (12 mg, 0.054 mmol, 1.0 equivalent) and triethylamine (8 ul, 0.06 mmol, 1.1 equivalent) in dry dichloromethane (1 ml) at 23 C. After 75 min, hydrogen peroxide (7 ul, 30% aqueous solution, 0.065 mmol, 1.2 equivalent) was added, and the reaction was stirred vigorously for 10 min and then quenched with sat Na2SO4 (1 ml). After 5 min of vigorous stirring, the solution was added to a mixture of dichloromethane (20 ml) and water (20 ml). The aqueous portion was extracted with dichloromethane (2×20 ml), and the combined organic fractions were dried (Na2SO4) and concentrated in vacuo. Flash chromatography (10 g SiO2; eluent 1% MeOH—CHCl3; product, fractions 12-15; 10 ml/fraction) afforded in addition to recovered ##STR5a## (2.5 mg, 21% yield), ##STR5b## (15 mg, 0.042 mm0l, 77% yield, 92% after two cycles) as a clear oil: Rf starting material, 0.78; product, 0.35 (5% MeOH-CHCl3, Verghns); [α]²³_{D}+25.40 (c=1.03, CHCl3); FTIR (film) 3079, 2934, 2880, 1647, 1451, 1269, 1240, 1161, 1003, 941 cm-1; sup.1H NMR (500 MHz, CDCl3) δ 5.74 (dd, 1H, J=10.3), 2.93-3.04 (m, 4H), 2.50-2.54 (m, 6H), 2.17-2.22 (m, 1H), 2.00-2.06 (m, 1H), 1.87 (s, 3H), 1.36-1.70 (m, 4H), 1.04 (s, 3H); sup.13 C NMR (101 MHz, CdCl3) δ 148.5, 146.9, 142.7, 114.6 (bm), 112.9, 110.4, 77.8 (bm), 58.7 (bm), 53.8, 47.3 (d), 41.7, 38.7, 33.8, 33.6, 27.9, 20.3, 18.3; sup.31 P NMR (121 MHz, CDCl3, Ph3P external standard at −6 ppm) δ 22.65 (t, J=10); HRMS (EI, Pos) m/z calculated for [C19H33O2N2P]+352.2280, found 352.2285.

### Example 7

Synthesis of ##STR6##

(−)-beta-elemene

A solution of dry ##STR5b## (53 mg, 0.152 mmol, 1.0 equivalent, azeotroped from toluene) and tert-amyl alcohol (67 ul, 0.608 mmol, 4.0 equivalent) in dry tetrahydrofuran (1.5 ml) was cannulated into a blue solution of excess lithium in liquid ammonia (5 ml) at −33 C. The transfer flask was rinsed with tetrahydrofuran (0.5 ml), and the solution was stirred for 10 h. The solution was sequentially quenched dropwise with isoprene (ca. 300 ul) and saturated aqueous NH4Cl (2 ml) and diluted with pentanes (4 ml). After warming to 23 C, the solution was added to a mixture of pentanes (2×30 ml), and the combined organic fractions were dried (Na2SO4) and concentrated in vacuo. Flash chromatography (10 g SiO2; eluent, pentanes; product, fractions 4-7; 10 ml/fraction) afforded ##STR6## (29.5 mg, 0.144 mmol, 95% yield) as a clear oil: Rf starting material, 0.00; product, 0.71 (petanes, Verghns); [α]²³_{D}−15.4° (c=0.59, CHCl3); FTIR (film) 3083, 2969, 2931, 1644, 1454, 1440, 1374, 1004, 909 cm-1; sup.1H NMR (500 MHz, CDCl3) δ 5.82 (dd, 1H, J=11.0, 17.4), 4.88-4.92 (m, 2H), 4.82 (t, 1H, J=1.6), 4.70-4.72 (m, 2H), 4.59 (bs, 1H), 1.99-2.03 (m, 1H), 1.92-1.96 (m, 1H), 1.75 (s, 1H), 1.71 (s, 3H), 1.42-1.63 (m, 6H), 1.01 (s, 3H); sup.13 C NMR (101 MHz, CDCl3) δ 150.4, 150.3, 147.7, 112.1, 109.8, 108.2, 52.8, 45.7, 39.9, 39.8, 32.9, 26.8, 24.7, 21.1, 16.6; HRMS (EI, Pos) m/z calculated for [C15H24]+204.1878, found 204.1869.

### Example 8

Synthesis of ##STR7##

(1S, 3R, 4R)-1-Acetyl-3-isopropenyl-4-methyl-4-vinylcyclohexane

A solution of (DHQD)2-PHAL (11 mg, 0.0137 mmol, 0.1 equivalent), potassium osmate (VI) dihydrate (0.5 mg, 0.0014 mmol, 0.01 equivalent), potassium ferrocyanide (135 mg, 0.411 mmol, 3.0 equivalent), potassium carbonate (57 mg, 0.411 mmol, 3.0 equivalent), and methanesulfonamide (13 mg, 0.137 mmol, 1.0 equivalent) in 1:1 2-methyl-2-propanol-water (1.5 ml) was cooled to 0 C. The biphasic mixture was added to ##STR6## (28 mg, 0.137 mmol, 1.0 equivalent) at 0 C and the reaction mixture was stirred for 11 h. The solution was quenched with excess Na2SO3 (until precipitate and color disappeared). After warming to 23 C, the solution was added to a mixture of dichloromethane (20 ml) and water (20 ml).

The aqueous portion was extracted with dichloromethane (2×20ml), and the combined organic fractions were dried (Na2SO4) and concentrated in vacuo. Flash chromatography (15 g of SiO2; eluent, 28% EtOAc-hexanes; product, fractions 19-30; 10 ml/fraction) afforded, in addition to recovered ##STR6## ( 5 mg, 0.024 mmol, 17% yield), a 3:1 mixture of diastereomers of the 1,2-diol (24.8 mg, 0.104 mmol, 76% yield) as a clear oil.

Sodium periodate (62 mg, 0.289 mmol, 3.0 equivalent) was added to a solution of the 1,2-diol (23 mg, 0.096 mmol, 1.0 equivalent) in 4:1 tetrahydrofuran-water (2 ml) at 23 C. After 30 min, the solution was added to a mixture of dichloromethane (20 ml) and water (20 ml). The aqueous portion was extracted with dichloromethane (2×20ml), and the combined organic fractions were dried (Na2SO4) and concentrated in vacuo. Flash chromatography (10 g of SiO2; eluent, 7% EtOAc-hexanes; product, fractions 3-9; 10 ml/fraction) afforded ##STR7## ( 19 mg, 0.092 mmol, 96% yield) as a clear oil: Rf starting material, 0.07; product, 0.61 (3:1 hexanes-EtoAc, Verghns); [α]²³_{D}+37.00 (c=1.0, CHCl3); FTIR (film) 3082, 2971, 2935, 2864, 1711, 1638, 1441, 1373, 1353, 908, 892 cm-1; sup.1H NMR (500 MHz, CDCl3) δ 5.80 (dd, 1H, J=10.6, 17.8), 4.89-4.93 (m, 2H), 4.84 (t, 1H, J=1.4), 4.60 (s, 1H), 2.37-2.43 (m, 1H), 2.16 (s, 3H), 1.97-2.00 (m, 1H), 1.74-1.78 (m, 1H), 1.67-1.71 (m, 5H), 1.46-1.59 (m, 3J), 1.00 (s, 3H); sup.13 C NMR (101 MHz, CDCl3) δ 211.6, 149.6, 146.9, 112.6, 110.3, 52.0, 51.9, 39.6, 39.1, 29.4, 28.2, 24.7, 23.7, 16.5; HRMS (EI, Pos) m/z calculate for [C14H22O]+206.1671, found 206.1661.

### Example 9

Synthesis of #STR8##

(+)-Fuscol

n-Butyllithium (244 ul, 1.57 M in hexanes, 0.384 mmol, 4.95 equivalengt) was added to a solution of 2-methyl-2-propanol (37 ul, 0.388 mmol, 5.0 equivalent) in tetrahydrofuran (0.5 ml) at −78 C. After 15 min, butyl (dibutylphosphono)-2-butenoate (108 ul, 0.388 mmol, 5.0 equivalent) was added, and the mixture was briefly warmed to effect solution. After 15 min at −78 C, the yellow phosphonate anoin solution was cannulated into ##STR7## (16 mg, 0.078 mmol, 1.0 equivalent) in tetrahydrofuran (0.5 ml) at 23 C. After 18 h, 5 equivalent of additional phosphonate anion was added in the same manner. This process was repeated at 28 and 41 h. After 48 h of stirring, the reaction mixture was diluted in dichloromethane, passed through silica gel (15 g, 200 ml CH2Cl2), and concentrated in vacuo. Flash chromatography (15 g of SiO2; eluent, 1.5% EtOAc-hexanes; product, fractions 7-15; 10 ml/fraction) afforded butyl

5-[(1′S,3′R,4′R)-3′-isopropenyl-4′-methyl-4′-vinylcyclohexyl]-(E,E)-hexadienoate (22.1 mg, 0.067 mmol, 87% yield) as a 12:1 mixture of diastereomers: Rf starting material, 0.55; product, 0.75 (5:1 hexanes-EtoAc, anisaldehyde). Preparative thin layer chromatography (0.5 mm plate, 9:1 pentanes-diethyl ether, Rf trans, trans-5-[(1′S,3′R,4 ′R) -3′-isopropenyl-4′-methyl -4′-vinylcyclohexyl]-(E,E) -hexadienoate, 0.42) afforded pure

5-[(1′S,3′R,4′R)-3′-isopropenyl-4′-methyl-4′-vinylcyclohexyl]-(E,E)-hexadienoate (80% yield) as a clear oil: [α]²³_{D}+24.50 (c=1.17, CHCl3).

Methyllithium (161 ul, 1.5 M in diethyl ether, 0.242 mmol, 5.0 equivalent) was added to a solution of

5-[(1′S,3′R,4′R)-3′-isopropenyl-4′-methyl-4′-vinylcyclohexyl]-(E,E)-hexadienoate (16 mg, 0.048 mmol, 1.0 equivalent) in diethyl ether (2 ml) at −30 C. After 12 h, the reaction was quenched with aqueous NH4Cl, warmed to 23 C, and added to a mixture of diethyl ether (10 ml) and water (10 ml). The aqueous portion was extracted with diethyl ether (2×20 ml), and the combined organic fractions were dried (Na2SO4) and concentrated in vacuo. Flash chromatography (15 g of SiO2; eluent, 6% EtOAc-1% triethylamine-hexanes; product, fractions 10-20; 10 ml/fraction) afforded ##STR2## ( 12.5 mg, 0.043 mmol, 90% yield) as a clear oil: Rf starting material, 0.75; product, 0.27 (5:1 hexanes-EtoAc, anisaldehyde); [α]²³_{D}+19.7° (c=1.0, CHCl3); FTIR (film) 3402, 3360, 3082, 2971, 2928, 2860, 1637, 1441, 1374, 966, 908, 890 cm-1; UV/vis λmax =240 nm, ε=35,000; sup.1 H NMR (500 MHz, CDCl3) δ 6.48 (dd, 1H, J=10.8, 15.3), 5.87 (d, 1H, J=10.8), 5.82 (dd, 1H, J=11.1, 17.2), 5.76 (d, 1H, J=15.3), 4.88-4.92 (m, 2H), 4.81 (t, 1H), J=1.5), 4.58 (s, 1H), 2.01 (dd, 1H, J=3.5, 12.6), 1.95-1.98 (m, 1H), 1.79 (s, 3H), 1.70 (s, 3H), 1.43-1.60 (m, 6H), 1.35 (s, 6H), 1.00 (s, 3H); sup.13 C NMR (126 MHz, CDCl3), δ 150.2, 147.6, 143.4, 139.3, 123.1, 122.3, 112.1, 109.9, 70.9, 52.8, 47.7, 39.9, 39.8, 32.7, 29.9, 26.6, 24.7, 16.7, 15.3; HRMS (EI, Pos) m/z calculated for [C20H32O]+288.2453, found 288.2440.

### Example 10

Synthesis of ##STR9## (Lr-1)

(R or S)-2 -((1R,3S,4S)-3-Isopropenyl-4-methyl-4-vinyl-cyclohexyl)-propane-1,2-diol ¹H NMR (400 Mhz ,CDCl3): δ=6.10 (1H, dd, J=17.6, 10.8 Hz), 5.15 (1H, d, J=18 Hz) 5.06 (1H, d, J=10.8 Hz), 4.70 (2H, s), 3.42 (1H, dd, J=11.2, 8.4 Hz), 3.23 (1H, dd, J=11.2, 5.2 Hz), 2.78 (1H, s), 2.15 (1H, dd, J=8.0, 5.2 Hz), 2.01 (1H, dd, J=12.4, 3.2 Hz), 1.93 (1H, tt, J=12.0, 3.2 Hz), 1.73 (3H, s), 1.61-1.56 (1H, m), 1.52-1.24 (5H, series of m), 1.26 (3H, s), 1.09 (3H, s).

### Example 11

Synthesis of ##STR10## (Lr-2)

(S)-2-((1R,3S,4S)-3-Isopropenyl-4-methyl-4-vinyl-cyclohexyl)-propane-1,2-diol, and (R)-2-((1R,3S,4S)-3-Isopropenyl-4-methyl-4-vinyl-cyclohexyl)-propane-1,2-diol ¹H NMR (400 Mhz ,CDCl3): δ=5.79 (1H, dd, J=17.6, 10.8 Hz), 4.81-4.91 (3H, m), 4.58 (s, 0.5 H), 4.56 (s, 0.5 H), 3.58 (1H, 1/2ABq, J=10.8 Hz), 3.43 (1H, 1/2ABq, J=10.8 Hz), 2.26 (1H, br s), 2.08 (1H, br s), 1.96 (1H, dd, J=12.4, 4.0 Hz), 1.70 (s, 1.5 H) and 1.69 (s, 1.5 H), 1.64-1.22 (7H, series of m), 1.14 (3H, s), 0.98 (3H, s).

### Example 12

Synthesis of ##STR11## (Lr-3)

1-((1R,3S,4S)-3-Isopropenyl-4-methyl-4-vinyl-cyclohexyl)-ethanone

¹H NMR (400 Mhz,CDCl3): δ=5.89 (1H, dd, J=17.6, 10.4 Hz), 4.91 (1H, d, J=13.6 Hz), 4.91 (1H, d, J=15.6 Hz), 4.84 (1H, s), 4.60 (1H, s), 2.46-2.36 (1H, s), 2.16 (3H, s), 1.99 (1H, dd, J=9.2, 7.2 Hz), 1.79-1.66 (2H, m), 1.71 (3H, s), 1.57-1.44 (4H, m), 0.99 (3H, s).

### Example 13

Synthesis of ##STRI2## (Lr-4)

(S)-1,5-Diisopropenyl-2-methyl-cyclohex-2-enol, and

(R)-1,5-Diisopropenyl-2-methyl-cyclohex-2-enol

¹H NMR (400 Mhz ,CDCl3): δ=1.55 (t, 1H, 2JHH=12.5 Hz), 1.61 (br s, 1H), 1.67 (s, 3H), 1.72 (s, 3H), 1.81 (s, 3H), 1.94 (m, 0.5H), 1.97 (m, 0.5H), 2.03 (m, 0.5H), 2.06 (m, 0.5H), 2.10 (m, 0.5H), 2.15 (m, 0.5H), 2.25 (m, 1H), 1.94 (m, 0.5H), 1.97 (m, 0.5H), 4.72 (s, 2H), 4.81 (s, 1H), 4.97 (s, 1H), 5.62 (s, 1H)

### Example 14

Synthesis of ##STR13## (Lr-5)

(S)-5-Isopropenyl-1,2-dimethyl-cyclohex-2-enol, and

(R)-5-Isopropenyl-1,2-dimethyl-cyclohex-2-enol

¹H NMR (400 Mhz ,CDCl3): δ=1.33 (s, 3H), 1.50 (bs, 1H), 1.66 (t, 1H, 2JHH=12.1 Hz), 1.74 (s, 6H), 1.89-1.98 (m, 2H), 2.09 (m, 1H), 2.30 (br t, 1H), 4.74 (s, 2H), 5.41 (s, 1H)

### Example 15

Synthesis of ##STR14## (Lr-6)

(S)-3,5-Diisopropenyl-2-methyl-cyclohex-2-enone

¹H NMR (400 Mhz ,CDCl3): δ=1.75 (br s, 6H), 1.88 (s, 3H), 2.29-2.70 (m, 5H), 4.76 (s, 2H), 4.81 (s, 1H), 5.05 (s, 1H)

### Example 16

Synthesis of##STR15## (Lr-7)

(1S,5S)-3,5-Diisopropenyl-2-methyl-cyclohex-2-enol, and

(1R,5S)-3,5-Diisopropenyl-2-methyl-cyclohex-2-enol

¹H NMR (400 Mhz ,CDCl3): δ=1.53 (t, 1H, xJHH=12.0 Hz), 1.65 (br s, 1H), 1.72 (s, 3H), 1.74 (s, 3H), 1.78 (s, 3H), 2.09-2.18 (m, 3H), 2.26 (br t, 1H), 4.18 (br t, 1H), 4.65 (s, 1H), 4.73 (s, 1H), 4.93 (s, 1H)

### Example 17

Synthesis of ##STR16## (Lr-8)

(1R,5S)-1-Isobutyl-3,5-diisopropenyl-2-methyl-cyclohex-2-enol, and

(1S,5S)-1-Isobutyl-3,5 -diisopropenyl-2-methyl-cyclohex-2-enol

¹H NMR (300 Mhz ,CDCl3): δ=0.92 (2,d, 3H), 0.99 (2,d, 3H), 1.54 (m, 2H) 1.72 (s, 3H), 1.74 (s, 3H), 1.83 (m, 1H), 1.87 (m, 1H), 1.95 (m, 1H), 2.10-2.18 (2 sets of m, 1H) 2.28 (br t, 1H), 4.74 (s, 2H), 5.37 (m, 1H)

### Example 18

Synthesis of##STR17## (Lr-9 and Lr-10)

(S)-5-Isopropenyl-2-methyl-cyclohex-2-enone, and

(R)-5-Isopropenyl-2-methyl-cyclohex-2-enone

¹H NMR (300 MHz ,CDCl3): δ=1.67 (s, 3H), 1.69 (s, 3H), 2.30-2.69 (m, 5H) 4.76 (s, 1H), 4.80 (s, 1H), 6.77 (br s, 1H)

### Example 19

Beta-elemene (2% Emulsion)'s inhibition of cancer cell growth in vitro. Elemene Injection (2.0%) demonstrated its ability to inhibit tumor growth in an in vitro study. Table 1 below summarizes these results. The results show that Elemene inhibits cancer cell growth and the IC₅₀ is lowest for HL-60 (human leukemia) and the highest for QGY (human liver cancer). Table 1 below.

| Cancer Cell Type | IC₅₀(ug/ml) |
|---|---|
| SHG-44 (human Glioma) | 60.47 |
| LAX (human lung cancer) | 92.80 |
| HL-60 (human leukemia) | 39.37 |
| QGY (human liver cancer) | 100.82 |
| MGC (human gastric cancer) | 83.64 |

### Example 20

Beta-elemene (2% Emulsion)'s efficacy against xenograph tumors in mice, axillary subcutaneous inoculation

In animal tests, Elemene is shown to inhibit tumor growth in certain types of cancer and extend animal survival time. Nude mice, C57BL/6 mice, and Kunming mice were used in animal studies. The cancer cell lines used were SHG-44 (human glioma tumor), LAX (human lung adenocarcinoma cells), G422 (mice brain tumor), and Lewis (lung cancer). Below only the results related to human brain tumor models were presented. Each experiment was repeated three times. Same sex animals were used across all treatment groups in each experiment. The three Elemene treated groups, E40, E20, and E10, were intravenously administered 40 mg/kg, 20mg/kg, 10 mg/kg Elemene Injection (2%) twice a day for 5 consecutive days, respectively. The positive control group received either CTX (cyclo-phosphate acylamine) at 30 mg/kg once a day for 7 consecutive days or VM-26 i.p. 5mg/kg once a day for 7 consecutive days. In addition, there was a placebo group that was administered placebo in the same manner as E40 group.

Axillary subcutaneous inoculation: Researchers took aseptically well-grown SHG-44 cells to prepare 1×10⁷/ml cell suspension. Nude mice were subcutaneously inoculated with 0.2 ml suspension. Treatment started the day after the inoculation. The animals were sacrificed 21 days after treatment (3 weeks). The tumors were excised and weighted. Table 2 summarizes the results of tumor inhibition rates (TIR) for SHG-44 human glioma (subcutaneous inoculation). A clear dose response was found in each of the three experiments. The TIR ranged from 41 % to 44% in the highest doses (E40) group and 28% to 33% in the lowest dose (E10) group. Each active treatment group was statistically significantly better than the placebo group.

TABLE 2
Summary of TIR for SHG-44 Human Brain Glioma (Subcutaneous Inoculation)

| Treatment | Tumor Weight (g) | | | TIR | | |
|---|---|---|---|---|---|---|
| (n) | Experiment 1 | Experiment 2 | Experiment 3 | Experiment 1 | Experiment 2 | Experiment 3 |
| E40 (6) | 1.12 ± 0.26* | 1.07 ± 0.27* | 1.08 ± 0.23* | 42.86 | 44.27 | 41.30 |
| E20 (6) | 1.18 ± 0.19* | 1.20 ± 0.14* | 1.18 ± 0.15* | 39.78 | 37.50 | 35.87 |
| E10 (6) | 1.35 ± 0.23* | 1.28 ± 0.17* | 1.32 ± 0.23* | 31.12 | 33.33 | 28.26 |
| CTX (6) | 0.33 ± 0.10* | 0.22 ± 0.04* | 0.23 ± 0.05* | 83.16 | 88.54 | 87.50 |
| Placebo (12) | 1.96 ± 0.28 | 1.92 ± 0.22 | 1.84 ± 0.25 | | | |

*P <= 0.01, compared with placebo control.
Tumor inhibition rate % (TIR) = (mean tumor weight in the control group − mean tumor weight in the treatment group)/(mean tumor weight in the control group) × 100

### Example 21

Beta-elemene(2% Emulsion)'s efficacy against xenograph tumors in mice, intracranial inoculation

Intracranial inoculation: Scientists took aseptically G422 tumor from well-growing mice to prepare 2×10⁷ cells/ml suspension by even protoplasm method. Mice were inoculated intracranially with 0.05 ml suspension. Treatment started the day after the inoculation. Animal survival time was recorded for 30 days. Table 3 summarizes the results of life prolongation rates (LPR) for G422 mice glioma (intracranial inoculation). A clear dose response was observed in each of the three experiments. The LPR ranged from 40% to 45% in the highest does group and 23% to 24% in the lowest dose group. All dose groups were significantly better than the placebo group.

TABLE 3
Summary of LPR for G422 Animal Glioma (Intracranial Inoculation)

| | Mean Survival Time (Days) | | | LPR | | |
|---|---|---|---|---|---|---|
| Treatment (n/m)⁺ | Experiment 1 | Experiment 2 | Experiment 3 | Experiment 1 | Experiment 2 | Experiment 3 |
| E40 (0/10) | 13.5 ± 3.8* | 15.1 ± 4.4* | 15.2 ± 4.8* | 56.07 | 57.29 | 59.16 |
| E20 (0/10) | 12.8 ± 3.4* | 14.0 ± 4.3* | 13.9 ± 4.9* | 47.98 | 45.83 | 45.55 |
| E10 (0/10) | 11.4 ± 3.9* | 12.6 ± 3.3* | 13.2 ± 4.8* | 31.79 | 31.25 | 38.22 |
| VM-26 (4/10) | 24.5 ± 6.0* | 25.8 ± 5.1* | 25.9 ± 4.7* | 183.24 | 168.75 | 171.2 |
| Placebo (0/20) | 8.65 ± 2.16 | 9.6 ± 1.88 | 9.55 ± 1.9 | | | |

⁺m and n denotes the number of animals alive at the beginning and the end of the treatment, respectively.
*P <= 0.01, compared with placebo control.
Life Prolongation Rate % (LPR) = (mean survival days in the control group − mean survival days in administered group)/(mean survival days in control group) × 100

According to animal testing, Elemene's effect in treating malignant brain tumor is similar to that of BCNU and VM26, which are the two most used drug in the market. However, Elemene has fewer side effects compared to BCNU and VM26. In addition, according to clinical research conducted in China, combination therapy of Elemene with BCNU or VM26 has better clinical effect than that of any single drug alone. The combination therapy could increase anti-tumor effect, and lower BCNU or VM26's side effects.

### Example 22

Protocol for animal tests for beta-elemene (2% Emulsion) and its intermediates and derivatives (elemene related).

Animal:

Use male athymic nude mice, at 4-5 weeks age, with weight at 18-20 g.

Tumor source: SHG44 glioblastoma cell line.

Procedure:

Animal work has been performed in the animal facility of Columbia medical School with institutional guidelines. Mice will be acclimated and housed in sterile cages in groups of four or less under laminar flow hoods in a temperature-controlled room with a 12-hour light/12-hour dark schedule, and fed autoclaved chow and water. Athymic nude mice will be planted with glioblastoma cells. For the cell implantations, glioblastoma cells grown in culture, will be washed with PBS, dispersed in a 0.05% solution of trypsin, and respuspended. After centrifugation (4000 rpm for 20 minutes at 8 C), the cell pellet will be suspended in PBS and the final concentration will be adjusted to 3×10⁷ cells/ml and suspension will be placed on ice. After the sites are cleaned with ethanol, 0.1 ml (3×10⁶ cells) of suspension will be subcutaneously injected in the right flanks of nude mice. Tumors will be measured with a dial-caliper, and the volumes are determined using the formular (width×length×height×0.52) (for ellipsoid form). After around 12 days, when the primary tumor is 1350-1500 mm³ in size, animals will be randomly divided into three groups: one treated with Elemene or elemene related (n=10), one negative (PBS) (n=10), and one positive (MMC) (n=10).

Dosage:

Yuanda's Elemene or elemene related i.p. at 80 mg/kg, once per day, 5 continuous days. Delivery method: i.p. once per day, 5 continuous days, total 5 times.

Control:

Negative control, i.p. PBS, same volume as Elemene i.p. once per day, 5 continuous days. Positive control: MMC (cytotoxic agent), i.p. 30mg/kg, once per day, 7 continuous days.

Tumor Weight Changes:

Use ruler to measure flank tumor size (3 groups) against time points as 24, 48, 72, 96, and 110 hrs starting from the first day of treatment.

### Example 23

Life span extension for animal studies

Pharmacological Animal Studies using Elemene injection (2% Emulsion).

Elemene injection (2% Emulsion) is given in 40, 20, or 10 mg/kg, twice daily, five days continuously, intravenous injection. The pharmacological results on animal brain tumor (implant into brain) G422 are as follows:
a. High dosage group, life span increases 56.07-59.16%;
b. Middle dosage group, life span increases 45.55-47.98%;
c. Low dosage group, life span increases 31.25-38.22%.
Tumor shrinkage is obvious.

Elemene is given at 80, 60, or 40 mg/kg through stomach (i.p.), the results are as follows:
a. High dosage group, life span increases 12.0-24.0%;
b. Middle dosage group, life span increases 35.1-37.0%;
c. Low dosage group, life span increases 30.9-36.1%.

Elemene is given through vein injection at 80 mg/kg, once daily, 10 days continuously, life span increases 52.60-54.17%, indicating that there is no obvious difference with same dosage but different injection route.

Elemene injection at 40, 20, 10 mg/kg, twice daily, five days continuously, results to human glioma SHG-44 (implant under the skin), Results:
a. High dosage group, tumor shrinkage 41.30-44.27%;
b. Middle dosage group, tumor shrinkage 35.87-39.78%;
c. Low dosage group, tumor shrinkage 28.26-33.33%.

Elemene injection at 40, 20, 10 mg/kg, twice daily, five days continuously. Results on lung cancer LAX model (implant through vein injection):
a. High dosage group, tumor shrinkage 40.00-44.94%;
b. Middle dosage group, tumor shrinkage 29.92-32.40%;
c. Low dosage group, tumor shrinkage 22.63-24.75%.

Elemene injection at 40, 20, or 10 mg/kg, twice daily, five days continuously, results towards Lewis lung cancer (implant through vein):
a. High dosage group, tumor shrinkage 37.74-40.64%;
b. Middle dosage group, tumor shrinkage 28.07-29.06%;
c. Low dosage group, tumor shrinkage 11.23-13.94%.

Normal Pharmacology Experiments

Elemene (2% Emulsion) could function as a peace drug, and it could increase sleeping pill's function in inhibit central nervous system. It has no obvious effect on mice and dog's blood pressure, heart rate, heart chart, and breathing rate.

Side effect, Partial Stimulating Effect

If Elemene is injected through the mice tail vein, the results are as follows.
a. Stimulating effect on little mice's tail vein blood vessel (light stimulating rate at 1.66);
b. Elemene's stimulating effect is less than its emulsion injection counterpart (median stimulating rate at 2.16).

Elemene injection through House rabbit's ear, its stimulating effects to rabbit's ear vein blood vessel is at median stimulating rate of 2.71, less than its emulsion version at Heavy stimulating rate of 3.78.

Thus for Elemene injection, its stimulating effect to blood vessel is 2.71, rated as median level stimulant. Its stimulating level is less than its emulsion injection version, which has a score at 3.78.

### Example 24

Toxicity Experiments using Elemene (2% Emulsion)

Acute Toxicity Test

Little Mice, injection through stomach, LD50=478.58 mg/kg

Injection through vein, LD50=189.76 mg/kg

Chronic Toxicity Test

Dogs and big mice are injected with Elemene through vein for three months at 6 mg/kg, with no toxic reaction, with no obvious change in blood, liver, and kidney.

The group injected at 12.5 mg/kg or 25 mg/kg has the following symptom:
a. no change in blood indication,
b. slight lower level of red blood cell, no changes in liver and kidney,
c. increases in red cell and white cell's levels under 400× microscope through normal urine test,
d. increases in urine protein,
e. no obvious changes in heart, brain, stomach, colon, kidney, or birth system,
f. Very small amount of animals have problems with liver, or kidney.

Pharmacokinetics Studies (Big Mice, ³H radioactivity test)

| | Total Radioactivity | Original Drug |
|---|---|---|
| Tβ | 1/212.8 +/− 2.9 h | 10.5 +/− 1.9 h |
| Bioavailability | 22.7% | 18.8% |

The drug's distribution is as follows:
a. Distrubution in the lung is very high.
b. Can pass blood brain barrier.
c. Can reach brain tumor part.

### Example 25

Beta-Elemene(2% Emulsion)'s efficacy against brain tumor in human patients Beta-Elemene drug has significant clinical benefit for brain tumor patients. In a clinical trial experiment conducted by Yuanda, Elemene drug is injected intra-arterially or intravenously (i.v.). The clinical trial was conducted from March 1999 to April 2001 at Chinese FDA designated hospitals. Among 39 glioblastoma patients in the trial, complete response (CR) is 5%, and partial response (PR) is 31%. Thus the overall tumor response rate is 36%. TEMODAR only has a CR+PR rate of 20%. In addition, 90% of the patients are relieved of the following symptoms: dizziness, headache, speech impairment, neurological dysfunction, and paralysis. Several patients complained of slight itching, which was relieved by hot patches. No allergic reactions were observed. No adverse reactions by liver, kidney, heart, stomach and GI tract, nerve system, and etc. No patient experienced severe lethal reactions. No vomiting or hematological abnormalities were observed.

Detailed description of the trial is as follows.

Clinical Testing Centers:
1. No. 1 University affiliated with China Medical University (lead hospital)
2. No. 1 University affiliated with Dalian Medical University
3. No. 2 University affiliated with Dalian Medical University

Clinical Testing Period:

March 1999-April 2001

Material and Methods

Drug

Pharmaceutical Company provides Elemene as a 2% Emulsion Injection Material, which is water-soluble. 200 mg/injection in a total volume of 10 ml. Label No. 990622 and 990715.

Patient Group

One test group, no control group. Comparison of CT or MRI scans of tumor before and after treatment.

Guidelines for Patient's Enrollment
1) Brain Tumor Patients diagnosed by CT or MRI, but not suitable for surgical removal of tumor.
2) Age: 18-70 years old.
3) Estimated survival: more than 3 months.
4) Relapsed after surgery.
5) Patients with large tumor inside the skull. Before treatment with Elemene, patients' tumors are first removed, but residue tumor still exists or patient relapsed after 2-4 weeks, shown by CT or MRI.
6) Patients who were or were not treated with chemotherapy, but showed no signs of improvement after 4 weeks of observation.
7) Patients can be clearly evaluated by CT or MRI.
8) No obvious heart, lung, liver, or kidney impairment.
9) No obvious history of allergy.

Drug Dosage and Method:
1) Glioblastoma Multiform.
2) Metastatic brain tumor.

Drug dosage and method (Clinical protocol):

Tumors at one side of the cerebrum:
Administered through carotid artery at the side of tumor:
Every other day injection. First, 600 mg,10% glucose is used to dilute the solution to half concentration (final volume 60 ml), adding 2 mg of Dexmethason (to prevent allergy and lessen side effects). Second 400 mg add into 10% glucose to a final volume of 500 ml, add 2.5 mg Dexmethason, intravenous drop through hand vein.
Administered not through carotid artery:
Every day 1000 mg in 1000 ml (final volume) 10% glucose injection, including 2.5-5 mg of Dexmethason.

Tumors at both sides of the cerebrum:
Injection through carotid artery at both sides of the brain. Left side: Monday, Wednesday and Friday; right side: Tuesday, Thursday, and Friday. Dosage as above.

Tumors at cerebellum hemisphere:
Mainly intravenous injection. Every day 1000 mg in 10% Glucose 1000 ml, adding 2.5-5 mg Dexmethason, slow drip. Finish intravenous injection in 5 hours, usually using tubes through vein under the collarbone or through neck vein.
Some people were given drug through spinal artery. Every time 600 mg, adding 2, mg Dexmethason, diluted in 10% Glucose to half concentration. Finish injection in 6-10 minutes. 1-2 times each week

Tumors at cerebrum, with obvious bulging bag.
Localized injection to the tumor bulging bag.
Use CT or MRI to locate the tumor bag position, suck out liquid (as much as possible), then inject 2% Elemene. The drug injection volume should be smaller than the liquid sucked out to avoid high pressure in the brain.
Pay attention to patients with high pressure in the brain.

Elemene dosage decreased for children.

Manna Alcohol Usage
Everyday 0.5-1 hour before drug use, intravenous drip 20% Manna Alcohol 250 ml, to open up Blood Brain barrier. This lowers down brain pressure, and improves treatment outcome.

Treatment Period
20-30 days. Highest total dosage is 30,000 mg, lowest 20,000 mg, or median 25,000 mg.

Patients expelled from the trial:

Patients who do not meet the guidelines in section 2.1

Other patients expelled from trial:

Dosage not optimum, treatment not as planned, in one month experienced with other chemotherapy or radiation, or accidental death during treatment.

Key Observations
1) Symptoms before and after treatment (including changes in body shape).
2) Blood indications before, during, and after treatment; changes in liver, kidney, and heart function before and after treatment.
3) Changes in CT or MRI of patients before and after treatment.
4) Adverse reaction during and after treatment.
   Efficacy and Toxicity Evaluation

Treatment Evaluation:
1) Symptoms and body shape change.
2) Tumor size change (CT or MRI).
Tumor size at 3-4 weeks after treatment is detected by CT or MRI, compared to that before treatment. Tumor volume is calculated, and Elemene's efficacy is evaluated according to WHO's international 5 level standard.

CR: Tumor disappears, and is unobservable beyond 4 weeks.

PR: Tumor size decreases more than 50%, and is unobservable beyond 4 weeks.

MR: Tumor size decreases between 25-50%.

SD: Tumor size decreases less than or not more than 25%, no new tumor appears.

PD: Tumor size increases more than 25% or new tumors appear.

Calculation Method:

Single Tumor Volume is calculated using standard method.

V=Tumor's largest dimension (L1)×largest dimension perpendicular to L1 (L2)×Tumor height/2

Tumor Shrinkage (%)=(((A−a)+(B−b)+(C−c) . . . )/(A+B+C+. . . ))×100% Note: A, B, C are Tumor volumes before treatment, a,b,c are tumor volumes after treatment.

Toxicities

According to WHO International anti-tumor drug normal toxicity classes, the toxicity standard is divided into four levels (I-IV): small, median, heavy, or life threatening level.

Results

Patient Base:

A total of 66 patients were enrolled. Of these 5 patients discontinued prematurely without providing post-baseline MRI/CT results (4 lost follow up and one died). These 5 patients were excluded from the analyses. Of the remaining 61 cases, 37 are male, and 24 are female, with an age spread of 8-79 years old (median age at 48.1 years old).

Disease Type:

39 cases of Glioblastoma and 22 cases of Metastatic brain tumor. In the 39 cases of Glioblastoma patients, 32 cases relapsed after one surgery, 2 cases relapsed after two surgeries.

Efficacy Evaluation

Symptom changes before and after treatment.

TABLE 3
Symptoms changes before and after treatment

| | Dizzi- | Head | | Speech | Neurological | Paraly- |
|---|---|---|---|---|---|---|
| | ness | Pain | Nausea | Impairment | Dysfunction | sis |
| Before | 2 | 49 | 24 | 7 | 12 | 12 |
| Treat- | | | | | | |
| ment | | | | | | |
| After | 0 | 3 | 2 | 0 | 0 | 5 |
| Treat- | | | | | | |
| ment | | | | | | |

Among the 61 cases, 25 patients are relieved of severe symptoms, at a recovery rate of 40.98%. All together 56 patients have obvious improvement in symptoms, at a rate of 91.8%.

Changes in tumor size.

According to WHO's five level evaluation chart, the results are as follows:

Percent reduction in tumor size: 48.75% (P<0.01).

CR: 9 cases—14.75%

PR: 15 cases—24.59%

MR: 9 cases—14.75%

SD: 27 cases—44.26%

PD: 1 case—1.6%

Tumor with somewhat shrinkage: 44 cases—72.1 %

Total CR+PR: 24 cases—39.34%.

Glioblastoma:

CR: 2 cases, PR: 12 cases. All CR+PR in Glioblastoma—35.90%.

Metastatic Brain Tumor:

CR: 7 cases, PR: 3 cases. All CR+PR in Metastatic Brain Tumor—45.45%.

Toxicity Side Effects

Several patients complained of slight itching (at the point where drug licks out), which was relieved by hot patches. No allergic reactions were observed. No adverse reactions by liver, kidney, heart, stomach and GI tract, nerve system, and etc. No patient experienced severe lethal reactions No vomiting or hematological abnormalities were observed.

TABLE 4
Results of Pain management by 2% Elemene.
Localized Pain could be divided into 4 categories.

| | No Pain | Glade I | Glade II | Glade III | Glade IV |
|---|---|---|---|---|---|
| During | 5 | 34 | 22 | 0 | 0 |
| Treatment | | | | | |
| After | 0 | 0 | 0 | 0 | 0 |
| Treatment | | | | | |

### Example 26

Beta-elemene (0.5% Emulsion)'s efficacy against lung cancer

Study 1—Combination Therapy with Injection Emulsion of Elemene and Radiation Therapy in the Treatment of Stage IV Nonsmall Cell Lung Cancer. Jie Li & Ju-Sheng Hou, The Cancer Hospital of the Second Affiliated Hospital, Dalian Medical University, Dalian, P. R. China.

| | Combination Therapy | Radiation Therapy |
|---|---|---|
| Patient Information | 30 patients with Stage IV | 30 patients with Stage IV |
| | non-small cell lung cancer. | non-small cell lung cancer. |
| Dosage | Elemene: 200-600 mg/m² for 2-4 | Radiation: Total - 40 Gy, 2 Gy per |
| | weeks (in some cases, 6 weeks). | session, 5 times per week. |
| | Radiation: Total - 40 Gy, 2 Gy | Dosage reduced to 24 Gy if signs |
| | per session, 5 times per week. | of tumor shrinkage are observed. |
| | Dosage reduced to 24 Gy if signs | For patients showing signs of |
| | of tumor shrinkage are observed. | metastasis to the bone, 30-40 Gy |
| | For patients showing signs of | total therapy is administered at 5 Gy |
| | metastasis to the bone, 30-40 Gy | intervals. For patients |
| | total therapy is administered at 5 Gy | showing signs of metastasis to the |
| | intervals. For patients | brain, a total dose of 30 Gy is |
| | showing signs of metastasis to the | administered at 1.5-2 Gy intervals |
| | brain, a total dose of 30 Gy is | (an additional 20 Gy is |
| | administered at 1.5-2 Gy intervals | administered if signs of tumor |
| | (an additional 20 Gy is | shrinkage are observed). |
| | administered if signs of tumor | |
| | shrinkage are observed). | |
| Delivery Method | Elemene: Continuous IV drip of | Radiation: Co⁶⁰ source. |
| | Elemene diluted in PBS for 2-4 | Localized radiation on tumor |
| | weeks. | and/or lymph nodes separately. |
| | Radiation: Co⁶⁰ source. | |
| | Localized radiation on tumor | |
| | and/or lymph nodes separately. | |
| Efficacy | CR: 6.6% | CR: 0% |
| | PR: 40% | PR: 23.3% |
| Side Effects | Decreased WBC count: level 1 | Decreased WBC count: level 1 |
| | (30%) and level 2 (3.3%). Two | (40%), level 2 (23.3%), level 3 |
| | patients dropped out of the study | (3.3%). Eight patients dropped |
| | because of reduced WBC counts. | out of study due to reduced WBC |
| | Nausea and vomiting: level 1 to 2 | counts. |
| | (16.7%), level 3 to 4 (3.3%). | Nausea and vomiting: level 1 to 2 |
| | Pneumonia due to radiation: 10% | (20%), level 3 to 4 (6.6%). |
| | Phlebitis: 16.7% | Pneumonia due to radiation: 10% |

Study 2—Comparative Study on Dual Artery Infusion of Elemene and Chemotherapeutic Agents in the Treatment of Lung Cancer. Xin Li, Shao-Xiong Xu, & Guo-Yan Shang, Department of Radiology, Guiyang Medical College, Guiyang, P. R. China.

| | Elemene Group | DAI Control Group | BAI Control Group |
|---|---|---|---|
| Patient Information | 30 lung cancer | 30 lung cancer | 30 lung cancer |
| | patients infused via | patients infused via | patients infused via |
| | DAI, along with | DAI, along with | BAI, along with |
| | Elemene and | chemotherapeutic | chemotherapeutic |
| | chemotherapeutic | agent alone. | agent alone. |
| | agent. | | |
| Dosage | Elemene: 500 mg/m² | Elemene: 500 mg/m² | Elemene: 500 mg/m² |
| | Standard | Standard | Standard |
| | chemotherapy drugs. | chemotherapy drugs. | chemotherapy drugs. |
| Delivery Method | 1^{st} treatment via BAI. | Same as Elemene | BAI 3-4 times. Drug |
| | 2^{nd} to 4^{th} treatment via | Group except that | mixture did not |
| | DAI (half BAI and | drug mixture did not | contain Elemene. |
| | half PAI) | contain Elemene. | |
| Efficacy | CR: 10% | CR: 6.7% | CR: 3.3% |
| | PR: 73.3% | PR: 70% | PR: 53.3% |
| | 1 and 2 year survival | 1 and 2 year survival | 1 and 2 year survival |
| | rate: 73.3% and 60%, | rate: 70% and 33.3%, | rate: 60% and 25%, |
| | respectively. | respectively, | respectively. |
| | Median survival time: | Median survival time: | Median survival time: |
| | 15 months | 12 months | 9 months |
| Side Effects | During BAI, patients | | |
| | complained of | | |
| | bronchial irritation, | | |
| | chest pains and | | |
| | coughs. Serious side | | |
| | effects included: | | |
| | anxiety, shortness of | | |
| | breath, cold sweats, | | |
| | breathing difficulties. | | |
| | Patients recovered | | |
| | after slowing injection | | |
| | rate and | | |
| | administration of | | |
| | Lidocain and | | |
| | Dexamethasone. | | |

BAI: Bronchial Artery Infusion
PAI: Pulmonary Artery Infusion
DAI: BAI & PAI

Study 3—Studies of Elemene Emulsion in Treating Late Stage Lung Cancer. Jia-liu Zhang, Xue-chang Zhang, & Jing-san Zhang, Department of respiratory system, Kunming No. 1 People's Hospital. Kunming, P. R. China.

| | Elemene Group |
|---|---|
| Patient | 11 late-stage lung cancer patients injected with |
| Information | Elemene alone. |
| Dosage | One treatment cycle: 400 mg Elemene in 250 ml |
| | of 5% GNS everyday for 10 days. |
| | One week break. |
| | 800 mg Elemene in 500 ml of 5% GNS everyday for |
| | 5 days. |
| | One week break. |
| | 800 mg Elemene in 500 ml of 5% GNS everyday for |
| | 5 days. |
| Delivery | IV drip. |
| Method | |
| Efficacy | CR: 0% |
| | PR: 50% |
| | PR (after 1 year): 40% |
| Side Effects | Phlebitis: 18.2%. Relieved by administration of |
| | 50 mg of Lidocain by IV before Elemene IV drip. |
| | Dexamethasone was also administered by IV after |
| | Elemene delivery. |
| | In the second treatment cycle, the injection device had |
| | to be buried in the vein due to hardening of the vein. |

Study 4—Clinical Trial Observation of Lung Cancer Patients Treated with Elemene Emulsion, Shu-kui Qin, Jun Qian, Lin Wang, & Ze-ming He, Department of Internal Medicine, Cancer Center, Nanjing Ba-yi Hospital, Nanjing, P. R. China

| | Elemene Therapy | rIL-11 Therapy |
|---|---|---|
| | (Primary Lung Cancer) | (Metastatic Lung Cancer) |
| Patient Information | 46 patients with median or late | 7 patients with median or late |
| | stage primary lung cancer | stage metastatic lung cancer |
| Dosage | 400 mg Elemene in 20 ml PBS, | 400 mg Elemene in 20 ml PBS, |
| | once a day, 10 days as a cycle. | once a day, 10 days as a cycle. |
| | 3 week break. Repeat once more. | 3 weeks break. Repeat once |
| | | more. |
| Delivery Method | IV | IV |
| Efficacy | CR: 4.3%, | CR: 0%, |
| | PR: 30.4%. | PR: 14.3%. |
| Side Effects | No change in liver and kidney | No change in liver and kidney |
| | function. | function. |
| | No change in electrocardiogram. | No change in electrocardiogram. |
| | Fever, phlebitis, nausea, | Fever, phlebitis, nausea, |
| | breathing irritation. | breathing irritation. |
| | 2 patients: coughed up blood. | Some patients experienced |
| | 1 patient: dramatic platelet | swelling, stuffiness, and heavy |
| | decrease and severe bleeding. | breath. The symptoms could be |
| | | relieved by slowing the injection |
| | | speed. |

### Example 27

Beta-elemene (0.5% Emulsion)'s efficacy against esophagus cancer and pancreatic cancer

Study 5—Clinical Evaluation of Elemene in Treating Esophageal Cancer and Pancreatic Cancer. Shi-yong Yang, Evaluation Group, on Elemene's Clinical Effect to esophagus cancer and pancreatic cancer patients, Xian, P. R. China

| | Esophagus Cancer | Pancreatic Cancer |
|---|---|---|
| Patient Information | 14 esophageal cancer patients. | 28 pancreatic cancer |
| | | patients. |
| Dosage | 300-600 mg Elemene, 5 days continuously. Repeat | |
| | treatment once more after 3 weeks. | |
| | Smallest dosage: 130 mg per session. | |
| | Largest dosage: 1000 mg per session. | |
| | Total treatment dosage: 61.9% in 3000-7000 mg | |
| | range. | |
| Delivery Method | 92.9% IV drip | |
| | 2 patients - IV under collarbone. | |
| | 1 patient - arterial injection under duodenum. | |
| Efficacy | CR: 0% | CR: 0% |
| | PR: 28.57% | PR: 25% |

| | |
|---|---|
| Side Effects | According to WHO's evaluation method, in both |
| | treatment groups: WBC level: 95.2% normal, |
| | 4.8% level I abnormal. |
| | Liver function (ALT): 95.2% normal, 2.4% at |
| | level I abnormal, 2.4% at level II abnormal. |
| | Kidney function (BUN): 100% normal. |

### Example 28

Beta-elemene (0.5% Emulsion)'s efficacy against gastrointestinal tract tumors

Study 6—Treatment of 30 Patients with Malignant Gastrointestinal Tract Tumors through Multi-method Delivery of Elemene. Qing-zhen Zhang, Li-xian Cu, & Xian-jun Zhu, Zhang-qiu People's Hospital, Zhang-qiu, P. R. China

| | Elemene Group | Chemotherapy Group |
|---|---|---|
| Patient Information | 30 patients with gastrointestinal | 28 patients with gastrointestinal |
| | tract tumors treated with | tract tumors treated with DPP |
| | Elemene alone. | and 5-FU. |
| Dosage | Elemene: 300 mg in 500 ml | Chemotherapy: 40 mg DPP on |
| | glucose solution once a day for 10 | days 1, 3, 8 and 10. 400 mg |
| | days continuously. | 5-FU from day 1 to 5. |
| | Additional oral intake of Elemene | Treatment cycle was repeated |
| | (100 mg) with 5 mg | after 3 weeks. |
| | Dexamethasone and 2 ml | |
| | Pu-lu-ka-yin in 10% glucose | |
| | solution. | |
| | Treatment cycle was repeated after | |
| | 3 weeks. | |
| Delivery Method | IV drip | DPP by IM, 5-FU by IV |
| Efficacy | CR: 36.6% | CR: 17.9% |
| | PR: 40% | PR: 28.5% |
| Side Effects | Fever: 2-6 hours after Elemene | |
| | injection, body temperature ˜38 C. | |
| | Patients recovered within one | |
| | week. | |
| | Oral intake of Elemene did not | |
| | result in adverse side effects | |
| | except for localized light pains. | |

Study 7—Clinical Effects of Elemene Emulsion in the Treatment of Late-Stage Gastrointestinal Tract Tumors through Induction of Stomach Ascites. Gui-fen Niu & Nan-sheng Cheng, Department of Digestive Systems, Suzhou No.2 People's Hospital, Suzhou, P. R. China

| | Elemene Group |
|---|---|
| Patient | 30 patients with late-stage gastrointestinal tumors |
| Information | |
| Dosage | 400 mg Elemene, 1-2 times each week, for 3 weeks. |
| | After 4 weeks, repeat the treatment cycle. |
| Delivery | Stomach ascites was first aspirate. Next, |
| Method | 20 ml 2% Lidocain was injected into stomach cavity, |
| | followed by 250 ml 0.9% PBS, and finally, 400 mg |
| | Elemene in 500 ml 0.9% PBS. |
| | This is called induced stomach ascites, which will |
| | be absorbed in 48 hours. |
| Efficacy | CR: 69.7% |
| | PR: 21.7% |
| Side Effects | Abdominal distension due to injection of drugs. |
| | Light stomach pain and stuffiness in the chest: 30%. |
| | Nausea and lack of appetite: 16.7%. |
| | No obvious changes in blood statistics |
| | No impairment of liver, kidney and cardiovascular function |
| | (measured by electrocardiograms). |

### Example 29

Beta-elemene (0.5% Emulsion)'s efficacy against colorectal cancer

Study 8—Clinical Effects Analysis of 65 Cases of Colorectal Cancer using Elemene Emulsion. Gao Xiang, Xue-zai Chen, & Gui-feng Chen, Department of Oncology, Nanpin No. 1 Hospital, Nanpin, Fujian Province, P. R. China

| | Elemene Group |
|---|---|
| Patient Information | 65 colorectal cancer patients. All patients |
| | had exercised surgical removal of colon between |
| | 6 months to 2 years ago. |
| Dosage | 400 mg Elemene, 4 times each week, for 6 months. |
| Delivery Method | Elemene was delivered (in the course of |
| | 1-2 hours) through the anus using inflatable devices |
| | which surround the drug delivery tube. |
| Efficacy | CR: 4.6% |
| | PR: 69.2% |
| Side Effects | Few side effects (no details). |

Study 9—Short-term Clinical Effect Observation of Late-Stage Colorectal Cancer Cases Treated by Elemene Emulsion through Conservative Enema. Qun-xiong Pan, & Jie-ji Guo, Department of Surgery, Quan-zhou No. 1 Hospital, Quanzhou, Fujian Province, P. R. China

| | Elemene Group | 5-FU Group |
|---|---|---|
| Patient | 17 late-stage colorectal cancer | 14 late-stage colorectal |
| Information | patients treated with Elemene | cancer patients treated |
| | alone. | with 5-FU. |
| Dosage | Elemene: 200 mg in 40 ml PBS | 5-FU: 500 mg 5-FU |
| | (incubated in colon for 2 hours) | in 40 ml PBS (incubated |
| | twice a day for 10 days | in colon for 2 hours) |
| | continuously. | twice a day for 10 days. |
| Delivery | Enema method | Enema method |
| Method | | |
| Efficacy | CR: 58.8% | CR: 57.1% |
| | PR: 23.5% | PR: 21.4% |
| Side Effects | No damage to heart, liver or | |
| | kidney. | |
| | No bone marrow inhibition. | |
| | No obvious reaction in digestive | |
| | system. | |

Study 10-18 Cases with Colon Obstruction after Colon Cancer Surgery Treated with Elemene Emulsion by Intravenous Injection Under Collarbone, Rui-lan Li, & Zhong-de Liu, Hunan Herbal Medicine Tumor Hospital, Changsha, Hunan Province, P. R. China

| | Elemene Therapy |
|---|---|
| Patient | 18 patients with colon obstruction after colon cancer |
| Information | surgery |
| Dosage | 400 mg Elemene in 100 ml PBS, once a day, 10 days as a |
| | treatment cycle. After 3 weeks of break, repeat the |
| | same cycle. |
| Delivery | IV injection under collarbone. |
| Method | |
| Efficacy | After 1 treatment cycle |
| | CR: 27.8% (no pain), PR: 44.4% (pain is relieved) |
| | After 2 treatment cycle |
| | CR: 22.2% (no colon blockage), PR: 44.4% (reduced colon |
| | blockage) |
| Side Effects | Fever (>38 C): 38.9%. Some over 39 C. |
| | Nausea and loss of appetite: 16.7%. |
| | No adverse effect on blood, liver and kidney function, |
| | and electrocardiogram |

### Example 30

Beta-Elemene (0.5% Emulsion)'s efficacy against stomach cancer

Study 11—Observation of Malignant Stomach Tumors Treated with Elemene Emulsion Through the Intestine, Jin-lian Zhang, & Mei-xia Wu, Fujian Longyan District No. 2 Hospital, Longyan, Fujian Province, P. R. China

| | Elemene Therapy | Elemene Therapy |
|---|---|---|
| | (Through Intestine) | (IV) |
| Patient | 15 malignant stomach tumor | 16 malignant stomach |
| Information | patients | tumor patients |
| Dosage | 300-400 mg Elemene in 100 ml | 300-400 mg Elemene |
| | 10% GS, 5-7 times per cycle. | in 100 ml 10% GS, |
| | Two cycles. | 5-7 times per cycle. |
| | | Two cycles. |
| Delivery | Intestinal injection (tube size | IV |
| Method | 10-15 cm) at 60-80 drops per | |
| | minute. Elemene left in intestine | |
| | for 2-4 hours after injection. | |
| Efficacy | CR: 6.7%, | CR: 0%, |
| | PR: 33.3%. | PR: 31.5%. |
| Side Effects | WBC decrease: 26.7% at level I, | WBC decrease: 37.5% |
| | 13.3% at level II | at level I, 6.25% at |
| | Frequent bowel movement: 2-4 | level II Phlebitis: 100%. |
| | times a day, recover on the | Hair loss, loss of |
| | second day, feces has liquid-like | appetite, and nausea. |
| | consistency. | |
| | Hair loss, loss of appetite, and | |
| | nausea. | |

### Example 31

Beta-elemen (0.5% Emulsion)'s efficacy against primary liver cancer

Study 12—Clinical Trial Summary on Primary Liver Cancer Patients Treated with Elemene Emulsion through Hepatic Arterial Injection, Li-seng Xiao, & Wei-ming Zhu, Wuxi No. 4 People's Hospital, Wuxi, Jiangsu Province, P. R. China

| | Elemene Therapy |
|---|---|
| Patient | 71 patients with primary liver cancer |
| Information | |
| Dosage | 400-1000 mg (mainly 600 mg) Elemene. |
| Delivery Method | Injection through hepatic artery and embolism. |
| Efficacy | CR: 2.8%, |
| | PR: 53.5%. |
| Side Effects | Fever: Some. |
| | Pain: 23.9% (level I), 5.6% (level II), 1.4% (level III). |
| | 1 patient: after treatment, experience shortness breath, |
| | stuffiness, swelling, palpitations, high blood pressure. |
| | These symptoms lasted 30 minutes and disappeared |
| | after proper treatment. |

### Example 32

The MDR cell line CEM/ADM 1.0 derived from sensitive parental cell line CEM overexpresses P170 glycoprotein on surface of cells. The experiment was divided into experimental groups, negative controls, and positive controls. When beta-elemene were used at 3 ug/ml, the dose modifying factor (DMF) on MDR CEM/ADM, which represent the IC50 without MDR modulator/IC50 with MDR modulator, were 1.84, 1.79, and 1.60 respectively for ADM, DAU, and VCR respectively by MTT method. At this concentration, the intracellular accumulations of ADM, DAU and VCR in CEM/ADM cells were some two times as much as those of negative controls. When beta-elemene was used at 4.5 ug/ml, the DMF of ADM, DAU and VCR on MDR CEM/ADM were 1.86, 1.79 and 1.72 respectively. Thus there was no significant deviation between both IC50 values for beta-elemene at 3 ug/ml or at 4.5 ug/ml (P>0.05). At this beta-elemene concentration, the intracellular accumulations of ADM, DAU and VCR in MDR CEM/ADM cells were about 2.6 times as much as those of negative controls.

When verapamil, a regular MDR reversion agent, was used at 7.5 ug/ml, the DMF of ADM, DAU, and VCR on MDR CEM/ADM were 2.02, 2.13, and 1.95 respectively. There was no significant deviation between both IC50 values (beta-elemene at 3 ug/ml and verapamil at 7.5 ug/ml, P>0.05). At this verapamil concentration, the intracellular accumulations of ADM, DAU and VCR in MDR CEM/ADM cells were about 2 times as nuch as those of negative controls. Beta-elemene showed no effect on IC50 values and drug accumulations in sensitive parental cell line CEM, and no quantity change of MDR1 mRNA in CEM.ADM cells by Rt-PCR.

Thus beta-elemene is able to reduce the IC50 of chemotherapeutic agents on MDR CEM/ADM cell line, but does not reverse to the levels of those compounds on sensitive parental cell. Beta-elemene may partially reverse the MDR effect in MDR CEM/ADM cell line. Its potency may be similar to that of verapamil on the same cell line.

### Example 33

The effect of beta-Elemene (2% Emulsion) on antitumor activity in human carcinoma cells was determined by the MTT survival assay, or using a commercial MTT assay kit (Cell Titer 96 Aqueous One Solution Cell Proliferation Assay; Promega Corporation, Madison, Wis.) according to the manufacturer's instructions. The MTT assay is a commonly used method in evaluation of cell survival, based on the ability of viable cells to convert MTT, a soluble tetrazolium salt [3-(4,5-dimethylthuazole-2-yl)-2,5 diphenyl tetrazolium bromide], into an insoluble formazan precipitate, which is quantitated by spectrophotometry following solubilization in dimethyl sulfoxide (DMSO).

In brief, carcinoma cells treated with beta-Elemene (2% Emulsion) alone, in 96-well tissue culture dishes were incubated with MTT (2 μg/ml) for 4 h. The cells were then solubilized in 125 μl of DMSO and absorbance readings were taken using a 96-well Opsys MRI Microplate Reader (ThermoLabsystems; Chantilly, Va.). The amount of MTT dye reduction was calculated based on the difference between absorbance at 570 nm and at 630 nm. Cell viability in treated cells was expressed as the amount of dye reduction relative to that of untreated control cells. The wells which contained only medium and 10 μl of MTT were used as blanks for the plate reader. Three sets of experiments were performed in 8-12 wells for each treatment.

Effect of Elemene on in vitro cytotoxicity in human cancer cells as assessed by the MTT assay

| Cancer cell type | Elemene IC₅₀ (ug/ml) | Elemene IC₅₀ (uM) |
|---|---|---|
| A-172 brain tumor | 65 | 32 |
| U-87 brain tumor | 88 | 43 |
| STTG1 brain tumor | 82 | 40 |
| NCI-H596 lung cancer | 95 | 47 |
| H-460 lung cancer | 32 | 16 |
| H-69 lung cancer | 52 | 25 |
| A2780/CP70 ovarian cancer | 53 | 26 |
| MCAS ovarian cancer | 60 | 29 |
| SKOV-3 ovarian cancer | 67 | 33 |
| ES-2 ovarian cancer | 54 | 26 |
| 5637 bladder cancer | 82 | 40 |
| T-24 bladder cancer | 65 | 32 |
| CCL-2 (Hela) cervical | 63 | 31 |
| cancer | | |
| HTB-33 cervical cancer | 68 | 33 |
| CCL-222 colon cancer | 47 | 23 |
| CCL-225 colon cancer | 67 | 33 |
| MCF-7 breast cancer | 93 | 46 |
| T47D breast cancer | 63 | 31 |
| DU-145 prostate cancer | 58 | 28 |
| PC-3 prostate cancer | 100 | 49 |

### Example 34

Anti tumor activities of LR1, LR2, and LR8 were evaluated by the MTT assay using human brain tumor, A-127, and the results are summarized below.

| | IC₅₀ (μg/ml) | | |
|---|---|---|---|
| Hour | LR1 | LR2 | LR8 |
| 24 | 176.24 | 131.78 | 106.2 |
| 48 | 163.23 | 89.60 | 105.2 |
| 72 | 133.13 | 90.90 | 103.3 |

### Example 35

The effect of beta-Elemene (2% Emulsion) and/or cisplatin on antitumor activity in human carcinoma cells was determined by the MTT survival assay, or using a commercial MTT assay kit (CellTiter 96 Aqueous One Solution Cell Proliferation Assay; Promega Corporation, Madison, Wis.) according to the manufacturer's instructions. The MTT assay is a commonly used method in evaluation of cell survival, based on the ability of viable cells to convert MTT, a soluble tetrazolium salt [3-(4,5-dimethylthuazole-2-yl)-2,5 diphenyl tetrazolium bromide], into an insoluble formazan precipitate, which is quantitated by spectrophotometry following solubilization in dimethyl sulfoxide (DMSO).

In brief, carcinoma cells untreated and treated cisplatin alone, or the combination of Elemene (2% Emulsion) (at IC20 of each cancer cell line) and cisplatin in 96-well tissue culture dishes were incubated with MTT (2 μg/ml) for 4 h. The cells were then solubilized in 125 μl of DMSO and absorbance readings were taken using a 96-well Opsys MRI Microplate Reader (ThermoLabsystems; Chantilly, Va.). The amount of MTT dye reduction was calculated based on the difference between absorbance at 570 nm and at 630 nm. Cell viability in treated cells was expressed as the amount of dye reduction relative to that of untreated control cells. The wells which contained only medium and 10 μl of MTT were used as blanks for the plate reader. Three sets of experiments were performed in 8-12 wells for each treatment.

Elemene increases cisplatin cytotoxicity and enhances cisplatin sensitivity in human cancer cells as determined by the MTT assay

| | | Cisplatin (uM) + | |
|---|---|---|---|
| Cancer cell type | Cisplatin IC₅₀ (uM) | Elemene | DMF |
| A-172 brain tumor | 24 | 0.25 | 96 |
| U-87 brain tumor | 10 | 1.8 | 5.6 |
| H-460 lung cancer | 80 | 8.0 | 10 |
| H-69 lung cancer | 8.0 | 1.5 | 5.3 |
| MCAS ovarian cancer | 38 | 6.5 | 5.8 |
| T-24 bladder cancer | 32 | 1.2 | 26.7 |
| CCL-2 (Hela) cervical | 27.5 | 3.0 | 9.2 |
| cancer | | | |
| HTB-33 cervical cancer | 32 | 3.8 | 8.4 |
| CCL-222 colon cancer | 32 | 3.5 | 9.1 |
| MCF-7 breast cancer | 28 | 0.38 | 73.7 |
| T47D breast cancer | 31 | 0.25 | 124 |
| DU-145 prostate cancer | 384 | 6.0 | 64 |
| PC-3 prostate cancer | 80 | 8.0 | 10 |

We claim:

1. A compound having the structure: ##STR6## (−)-beta-elemene
wherein R is selected from the group consisting of hydrogen, methyl, ethyl, n-propyl, n-hexyl, CO.sub.2 Et, CH.sub.2 OH, (CH.sub.2).sub.3 OH, and ##STR7##
and wherein R′ and R.sub.0 are each independently selected from the group consisting of linear or branched alkyl, substituted or unsubstituted alkoxy alkyl, substituted or unsubstituted alkoxy carbonyl, substituted or unsubstituted aryloxyalkyl, substituted or unsubstituted aroyl or benzoyl, trialkylsilyl, diarylalkylsilyl, aryldialkylsilyl, and triarylsilyl.

2. A compound having the structure: ##STR2##
wherein R is hydrogen or methyl; and wherein R′ and R.sub.0 are each independently selected from the group consisting of linear or branched alkyl, substituted or unsubstituted alkoxyalkyl, substituted or unsubstituted alkoxycarbonyl, substituted or unsubstituted aryloxyalkyl, substituted or unsubstituted aroyl or benzoyl, trialkylsilyl, diarylalkylsilyl, aryldialkylsilyl, and triarylsilyl.

3. Two de novo synthesis routes of ##STR6##.

4. The discovery of the unexpectedly efficacious, safe, non-multi-drug resistant effect, non-toxic, and broadly applicable use of (−)-beta-elemene as an anti-viral, anti-microbial, anti-biotic and especially as an anti-cancer chemotherapeutic.

5. (−)-beta-elemene, (−)-beta-elemene derivatives (##STR9## to ##STR17##) and (−)-beta-elemene-like structures are claimed, as are the processes by which said structures are obtained as well as the processes by which (−)-beta-elemene is obtained.

6. The use of (−)-beta-elemene and (−)-beta-elemene derivatives and (−)-beta-elemene-like structures formulated singularly or in combination for anti-viral, anti-microbial, and anti-cancer applications.

7. Pharmaceutical composition comprising (−)-beta-elemene derivatives and (−)-beta-elemene-like structures with all available pharmaceutical carriers.

8. The chemical in claim 1 is effective against brain tumor, lung cancer, colorectal cancer cancer, gastric intestinal cancer, and stomach cancer.

9. A method of treating cancer in a subject comprising: administering a therapeutically effective amount of (−)-beta-elemene and (−)-beta-elemene derivatives and (−)-beta-elemene-like structures to a subject in need thereof.

10. The method of claim 9, wherein the therapeutically effective amount of beta-elemene is between 3 mg/kg to 300 mg/kg.

11. (−)-beta-elemene and (−)-beta-elemene derivatives and (−)-beta-elemene-like reverse Multi-drug Resistance (MDR) effect in cancer cells.

12. A method of treating at least one cell line of cancer in a mammalian patient, comprising the following steps;
combining in a pharmaceutically acceptable carrier a therapeutically effective amount of cisplatin within the therapeutic window for cisplatin, and a therapeutically effective amount of (−)-beta-elemene within the therapeutic window for (−)-beta-elemene, to treat cancer patients, and administering said cisplatin and (−)-beta-elemene to said mammalian patient to achieve a therapeutically effective change in progression of said at least one cancer cell line, such as brain tumor, lung cancer, ovarian cancer, bladder cancer, cervical cancer, colon cancer, breast cancer, and prostate cancer.

13. A method of treating at least one cell line of cancer in a mammalian patient, comprising the following steps;
combining in a pharmaceutically acceptable carrier a therapeutically effective amount of cisplatin within the therapeutic window for cisplatin and a therapeutically effective amount (−)-beta-elemene derivatives and (−)-beta-elemene-like structures within the therapeutic window for (−)-beta-elemene derivatives and (−)-beta-elemene-like structures to treat cancer patients, and administering said cisplatin and (−)-beta-elemene derivatives and (−)-beta-elemene-like structures to said mammalian patient to achieve a therapeutically effective change in progression of said at least one cancer cell line.

14. A method of treating at least one cell line of cancer in a mammalian patient, comprising the following steps;
combining in a pharmaceutically acceptable carrier a therapeutically effective amount of Taxol within the therapeutic window for Taxol, and a therapeutically effective amount of (−)-beta-elemene within the therapeutic window for (−)-beta-elemene, to treat cancer patients, and administering said Taxol and (−)-beta-elemene to said mammalian patient to achieve a therapeutically effective change in progression of said at least one cancer cell line, such as brain tumor, lung cancer, ovarian cancer, bladder cancer, cervical cancer, colon cancer, breast cancer, and prostate cancer.

15. A method of treating at least one cell line of cancer in a mammalian patient, comprising the following steps;
combining in a pharmaceutically acceptable carrier a therapeutically effective amount of Taxol within the therapeutic window for Taxol and a therapeutically effective amount (−)-beta-elemene derivatives and (−)-beta-elemene-like structures within the therapeutic window for (−)-beta-elemene derivatives and (−)-beta-elemene-like structures to treat cancer patients, and administering said Taxol and (−)-beta-elemene derivatives and (−)-beta-elemene-like structures to said mammalian patient to achieve a therapeutically effective change in progression of said at least one cancer cell line.

16. A method of treating at least one cell line of cancer in a mammalian patient, comprising the following steps;
combining in a pharmaceutically acceptable carrier a therapeutically effective amount of 5FU within the therapeutic window for 5FU, and a therapeutically effective amount of (−)-beta-elemene within the therapeutic window for (−)-beta-elemene, to treat cancer patients, and administering said 5FU and (−)-beta-elemene to said mammalian patient to achieve a therapeutically effective change in progression of said at least one cancer cell line, such as brain tumor, lung cancer, ovarian cancer, bladder cancer, cervical cancer, colon cancer, breast cancer, and prostate cancer.

17. A method of treating at least one cell line of cancer in a mammalian patient, comprising the following steps;
combining in a pharmaceutically acceptable carrier a therapeutically effective amount of 5FU within the therapeutic window for 5FU and a therapeutically effective amount (−)-beta-elemene derivatives and (−)-beta-elemene-like structures within the therapeutic window for (−)-beta-elemene derivatives and (−)-beta-elemene-like structures to treat cancer patients, and administering said 5FU and (−)-beta-elemene derivatives and (−)-beta-elemene-like structures to said mammalian patient to achieve a therapeutically effective change in progression of said at least one cancer cell line.
